(19) United States
(12) Patent Application Publication
(10) Pub. No.: US 2004/0158884 A1
(43) Pub. Date: Aug. 12, 2004

(54) Transgenic mouse having conditional knock-out of myostatin function

(76) Inventors: Luc Grobet, Esneux (BE); Michel Geroges, Villers-aux-Tours (BE); Dimitri Pirottin, Sprimont (BE)

Correspondence Address: MCHALE & SLAVIN, P.A., 2855 PGA BLVD, PALM BEACH GARDENS, FL, 33410, US

(21) Appl. No.: 10/360,525

(22) Filed: Feb. 6, 2003

(51) Int. Cl. A01K 67/027; C12N 5/06

(52) U.S. Cl. 800/18; 435/354

(57) ABSTRACT

The present invention involves carrying out a gene targeting method utilizing the cre-lox system in order to generate transgenic mice allowing for conditional inactivation of the myostatin gene. The transgenic mice of the present invention that express a conditionally inactivated myostatin gene also exhibit a phenotype characterized by skeletal muscle hypertrophy.

### CROSS REFERENCE TO RELATED APPLICATIONS

This application is related to the following applications: Ser. No. 08/891,789, filed on Jul. 14, 1997, now U.S. Pat. Nos. 6,103,466; 09/007,761, filed on Jan. 15, 1998, now abandoned; PCT/IB98/01197, filed on Jul. 14, 1998; Ser. No. 09/482,573, filed on Jan. 13, 2000, now pending and Ser. No. 10/251,115, filed on Sep. 20, 2002, now pending, the contents of which are each herein incorporated by reference.

### FIELD OF THE INVENTION

The instant invention relates to the field of molecular genetics, in particular to the generation of transgenic mice, and most particularly to the generation of conditional knock-out mice which allows for the study of myostatin gene regulation at different stages of development.

### BACKGROUND OF THE INVENTION

Myostatin is a member of the transforming growth factor β (TGFβ) superfamily of secreted growth and differentiation factors essential in regulating the fate and behavior of tissues in early embryogenesis (McPherron et al. Nature 387:83-90 1997). All members of this superfamily share a common structure including a short peptide signal for secretion and an N-terminal peptide fragment that is separated from the bioactive carboxy-terminal fragment by proteolytic cleavage at a highly conserved proteolytic cleavage site. The myostatin gene is composed of three exons. The bioactive carboxy-terminal domain lies with the third exon and is characterized by cysteine residues at highly conserved positions which are involved in intra- and intermolecular disulfide bridges. The functional myostatin protein molecules are covalently linked (via a S-S bond) dimers of the carboxy-terminal domain. Myostatin is expressed in skeletal muscle and its precursors from early embryonic stages until adulthood. Myostatin expression is also observed at a lower level in adipose tissue (McPherron et al Nature 387:83-90 1997). Myostatin mRNA was observed in the mammary gland (Ji et al. American Journal of Physiology 275:part 2, R1265-1273, 1998) and in cardiac muscle (Sharma et al. Journal of Cell Physiology 180:1-9 1999).

Constitutive loss of mysostatin function results in a dramatic increase in skeletal muscle mass as a result of combined muscle hyperplasia and hypertrophy. Both myostatin knock-out mice along with (McPherron et al. Nature 387:83-90 1997) mice (Szabo et al. Mammalian Genome 9:671-672 1998 and Varga et al. Genetics 147:755-764 1997) and cattle (Grobet et al. Nature Genetics 17:71-74 1997; Grobet et al. Mammalian Genome 9:210-213 1998; Kambadur et al. Genome Research 7:910-915 1997 and McPherron et al. PNAS USA 94:12457-12461 1997) which are homozygous for naturally occurring myostatin loss-of-function mutations share this phenotype commonly referred to as “double-muscling”. More recently, transgenic mice that constitutively over-express dominant negative myostatin alleles under the dependence of strong skeletal muscle specific promoters were shown to be “double-muscled” as well (Lee et al. PNAS USA 98:9306-9311 2001; Yang et al. Molecular Reproductive Development 60:351-361 2001 and Zhu et al. FEBS Letters 474:71-75 2000). Additionally, over-expression or an excess of myostatin causes wasting in mice (Zimmers et al. Science 296:1486-1488 2002).

While both the transgenic mice (McPherron et al. Nature 387:83-90 1997) disclosed in the prior art and the transgenic mice of the instant invention exhibit reduced or completely disrupted expression of myostatin, the transgenic mice of the instant invention allow for reduced or completely disrupted expression at a desired time period, for example, but not limited to, later stages of development.

The fact that in nine out of eleven European cattle breeds double-muscling is due to five independent disruptive mutations in the same gene indicates that the number of genes affecting muscular development is likely to be limited (Grobet et al. Mammalian Genome 9:210-213 1998 and Capuccio et al. Proceedings of the XXVI International Conference on Animal Genetics, ISAG, Aug. 9-14, 1998, Auckland, New Zealand). However little is currently known about the molecular mechanisms by which myostatin is able to regulate the skeletal muscle mass. Expression of myostatin during the entire lifetime of an organism, and in particular after birth, could mean that myostatin retains, at least partially, its regulating properties over a long period. Moreover, several studies suggest that postnatal changes in myostatin expression could be associated with, if not causative of, skeletal and cardiac muscle depletion or regeneration (Carlson et al. American Journal of Physiology 277:part 2, R601-606 1999; Casas et al. Journal of Animal Science 77:1686-1692 1999; Gonzales-Cadavid et al. PNAS USA 95:14938-14943 1998; Sharma et al Journal of Cell Physiology 180:1-9 1999 and Bogdanovich et al. Nature 420:418-421 2002). However it remains unknown whether the inhibition of myostatin expression at later stages of development will retain the potential to promote muscle growth. If a methodology and a research tool could be devised which would aid in answering this question, it would enhance the development and administration of a myostatin antagonist for the treatment of muscle wasting or as a means to enhance meat production of farm animals.

### SUMMARY OF THE INVENTION

The instant invention provides such a research tool in the form of transgenic mice which allow for conditional inactivation of the myostatin gene at later stages of development. The transgenic mouse of the instant invention can be used to aid in development of means to treat disease conditions of the muscular-skeletal system. The instant invention involves carrying out a gene targeting method utilizing the cre-lox system. As shown in FIG. 1, the third exon of the myostatin gene (which encodes the bioactive domain) is floxed (flanked with loxp sites) allowing its excision conditional on the expression of cre recombinase. After the expression of cre recombinase, the third exon is deleted generating a null allele. The transgenic mice of the instant invention which are homozygous for the expression of a conditionally inactivated myostatin gene also exhibit a phenotype characterized by skeletal muscle hypertrophy.

Accordingly, it is an objective of the instant invention to provide a mouse embryonic stem cell comprising a floxed myostatin allele that can be used to produce a transgenic mouse having a floxed myostatin allele.

It is another objective of the instant invention to provide a transgenic mouse comprising a myostatin gene wherein exon 3 of said myostatin gene is floxed and can be conditionally inactivated with excision of exon 3 by cre recombinase.

It is another objective of the instant invention to provide a method for producing a transgenic mouse having a myostatin gene conditionally inactivated.

It is yet another objective of the instant invention to provide a transgenic mouse having a conditionally inactivated myostatin gene; said mouse also exhibits a phenotype characterized by muscular hypertrophy.

Other objectives and advantages of this invention will become apparent from the following description (including the experimental working examples) taken in conjunction with the accompanying drawings wherein are set forth, by way of illustration and example, certain embodiments of this invention. The drawings constitute a part of this specification and include exemplary embodiments of the instant invention and illustrate various objects thereof.

As used herein, the abbreviation “MSTN” means “myostatin”.

As used herein, the abbreviation “ES” means “embryonic stem”.

As used herein, the term “cre recombinase” refers to a specific DNA recombinase which recognizes a specific nucleotide sequence (lox site) and conducts complete processing, including strand cleavage, strand exchange and ligation of each strand within the site. A cre gene can be isolated from the E. coli bacteriophage P1 by methods known in the art (Abremski et al. Cell 32:1301-1311 1983; Sternberg et al. Journal of Molecular Biology 150:467-486 1981). The use of a Cre/lox system provides specific gene expression at a specific desired time.

As used herein, the term “lox” refers to a specific sequence of nucleotides recognized by cre recombinase. There are several different lox sites, for example, loxp, loxB, loxl, loxR and loxC2. These sequences can be isolated from the E. coli bacteriophage P1 by methods known in the art (Hoess et al. PNAS USA 79:3398 1982; Sternberg et al. Journal of Molecular Biology 150:487-507 1981). The preferred lox site used in the methods of the instant invention is the loxP site. LoxP is a 34 base pair nucleotide sequence (positions 7-40 of SEQ ID NO:21; positions 8-41 of SEQ ID NO:23; positions 8-41 of SEQ ID NO:25; see FIG. 5) consisting of two 13 base pair inverted repeats separated by an 8 base pair spacer region.

As used herein, the term “flox” means to flank a portion of a nucleotide sequence (gene) with one or more loxP sites.

### BRIEF DESCRIPTION OF THE FIGURES

This patent or application file contains at least one drawing executed in color (FIG. 3). Copies of this patent or patent application publication with color drawing(s) will be provided by the Office upon request and payment of the necessary fee.

FIG. 1 shows the third exon of the myostatin gene flanked by LoxP sequences.

FIG. 2 shows target sites for the introduction of LoxP sequences (A and B).

FIG. 3 shows an overview of the engineering strategy.

FIG. 4 shows the expected sizes of the fragments obtained after digestion of the SalI murine insert with BssSI and BsaI.

FIG. 5 shows the nucleotide sequences (numbered from top through bottom SEQ ID NO:7-SEQ ID NO:26 respectively) of the adaptors used during the engineering of the murine clone.

FIG. 6 shows a schematic representation of pPonc1b.

FIG. 7 shows a schematic representation of pPonc2b.

FIG. 8 shows a schematic representation of pPonc3j.

FIG. 9 shows the final construct for use in homologous recombination in ES-cells.

FIG. 10 shows the killing curve of the parental CHO-K1 cell line incubated with increasing concentrations of neomycin.

FIG. 11 shows the two types of deletions found in ES cells after transient expression of the Cre recombinase and selection for the neomycin sensitive clones.

FIG. 12 shows the composition of the targeting construct.

FIG. 13 shows a schematic representation of a cross-section of the widest part of the lower leg.

FIG. 14 shows detection of MSTN transcripts by RT-PCR in a range of tissues from two-month old mice of four different MCKcre^{./.} MSTN^{./.} genotypes.

FIG. 15 shows live weight at five months (g), carcass weight (g) and weight of the pectoralis muscles (g) in the MCKcre^{+/-} MSTN^{+/flox} intercross.

FIG. 16 shows cre-mediated excision of the MSTN third exon.

FIGS. 17A-C show a comparison between a MCKcre^{+/?} MSTN^{+/+} and MCKcre^{+/?} MSTN^{flox/flox} individual, (A) the thoracic portion of the carcass, (B) the caudal portion of the carcass and (C) the hematoxylin-eosin stained cross sections of the lower leg (see FIG. 13 for a schematic of the cross-section of the lower leg).

FIG. 18 shows frequency distribution of myofibres with a given cross-sectional area in the tibialis cranialis and gastrocnemius plantaris muscle groups, for the MCKcre^{+/?} MSTN^{+/+} (white) and MCKcre^{+/?} MSTN^{flox/flox} (black) genotypes.

FIG. 19 shows Table 3.

FIG. 20 shows Table 4.

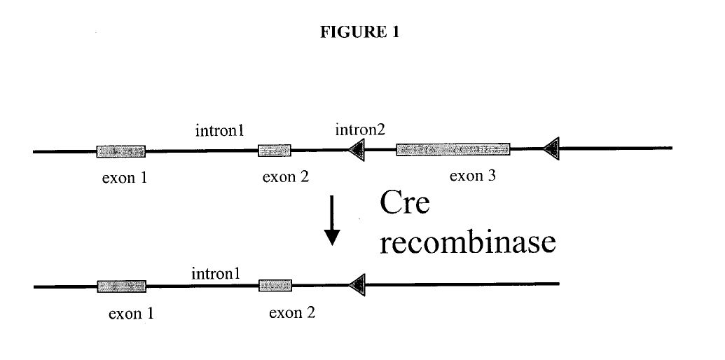

### DETAILED DESCRIPTION OF THE INVENTION

A transgenic animal, for example, a mouse, is an animal having cells that contain a transgene, which transgene is introduced into the animal or an ancestor of the animal at a prenatal stage, for example, an embryonic stage. A transgene is a nucleotide sequence which is integrated into the genome of a cell from which a transgenic animal is developed. Various types of nucleotide sequences can be used to generate transgenic animals, for example, mutant sequences and heterologous sequences. “Knock out” animals can also be generated, such as the mice of the instant invention, wherein entire genes or parts of genes are deleted or “knocked-out” to discern function.

Methods for generating transgenic animals, particularly animals such as mice, have become conventional in the art and are described, for example, in U.S. Pat. No. 4,736,866.

The conditional knock-out mouse of the instant invention is of major interest to address questions of concerning the effect of spatio-temperal inactivation of myostatin in mammals since the inactivation can be controlled. It has been previously demonstrated that a site specific excision using the Cre-LoxP recombination system works with high efficiency both in ES cells and a mammalian organism when the cre recombinase gene is properly expressed (Gu et al. Cell 73:1155-1164 1993; Gu et al. Science 265:103-106 1994; Meyers et al. Nature Genetics 18:136-141 1998). The Cre-LoxP recombination strategy of the instant invention eliminates the neomycin resistance cassette from the floxed myostatin gene at the ES cell stage after transient expression of cre recombinase (see FIG. 11). In FIG. 11, Type I (dashed lines) recombination event results in a non-functional myostatin allele and Type II recombination produces the floxed functional allele. This deletion limits the foreign insertion to the two LoxP sites (34 base pairs each) in the floxed allele which allows for normal expression of the encoded myostatin.

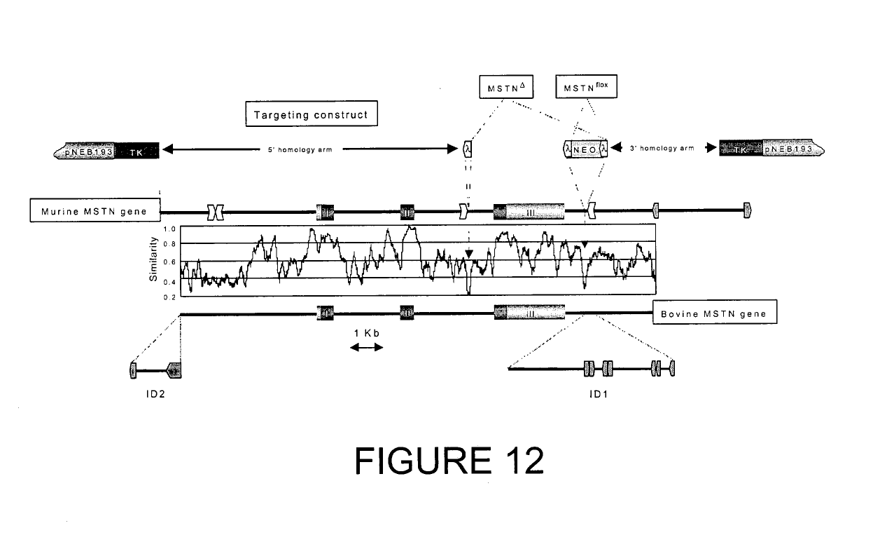

The first stage in the preparation of the mouse of the instant invention is the engineering of a murine floxed myostatin allele for homologous recombination in embryonic stem cells. In order to generate a MSTN allele that would allow for its conditional inactivation, the third exon of the myostatin gene (known to encode the bioactive carboxyterminal domain) was flanked with a pair of loxP sites. To prevent disruption of putative cis acting elements, the loxP sites were inserted in regions of low similarity with the bovine orthologous sequences. These sequences of low similarity were identified by alignment of the bovine and murine MSTN gene sequences using the algorithm of Needleman and Wunsch (Journal of Molecular Biology 48:443-453 1970) implemented with the BESTFIT program (GCG Wisconsin Package™). Two major inserts present in the bovine but not in the murine sequence and detected by dotplot analysis (FIG. 12) were eliminated before performing the BESTFIT alignment. A similarity profile was generated by sliding a 200 base pair window through the aligned sequences and computing the percentage similarity between the bovine and murine sequences for each window (FIG. 12). The targeting vector was constructed using standard cloning procedures (Sambrook and Russel Molecular Cloning. A laboratory manual. Third Edition, Cold Spring Harbor Laboratory Press, 2001) and is schematically represented in FIG. 12. In FIG. 12, the murine and bovine MSTN genes are aligned. The three exons (I, II and III) are represented as cylinders with the 5′ and 3′ UTR sequences shown in light and the coding sequences shown in dark. The dotted arrows correspond to SINE sequences. “ID1” and “ID2” correspond to insertion/deletion events present in the bovine sequence but not in its murine orthologue. A similarity profile, corresponding to the sequence similarity of a 200 base pair window slided across the aligned murine and bovine sequences is plotted. The approximate positions in the targeting construct of the two thymidine kinase cassettes (TK), the neomycin resistance cassette (NEO), the three loxP sites (λ), the 5′ and 3′ homology arms, and the vector sequences (pNEB193) are shown. The vertical arrows point towards the similarity dips in which the floxed neomycin resistance cassette and the isolated 5′ loxP site were inserted. The location of the two primer pairs used in the assay to moniter cre-mediated excision of the third exon are shown as white arrows. The deletions obtained by transient expression of cre in R1 embryonic stem cells and yielding the MSTN^{Δ} and MSTN^{flox} alleles are shown. To summarize, the targeting vector comprises (i) a loxP site in a poorly conserved region of intron 2, (ii) a floxed neomycin resistance cassette inserted in a poorly conserved region 3′ of the main polyadenylation site, (iii) 8.2 and 3.3 Kb of 5′ and 3′ homology arms, respectively, and (iv) two thymidine kinase selection cassettes at either end of the construct.

An overview of the building blocks for the vector construction are:

(i) a lambda clone (λ-MMMSTN-1) containing the entire murine MSTN gene plus 4 and 6.5 Kb of upstream and downstream sequences. This clone was isolated from a murine genomic library (ML1043J-Clontech, Palo Alto, Calif.) constructed from C57B16/6N genomic DNA. The 15,382 base pair insert of the λ-MMMSTN-1 has been completley sequenced and submitted to GenBank under the accession number AY204900.

(ii) a cassette containing a bacterial neomycin phosphotranferase gene under the dependence of the thymidine kinase promoter, isolated from the pMC1neoPolyA vector (Stratagene, La Jolla, Calif.).

(iii) a cassette containing the herpes simplex thymidine kinase gene under the dependence of the CMV promoter isolated from the pcDNA3 vector (Invitrogen, Carlsbad, Calif.).

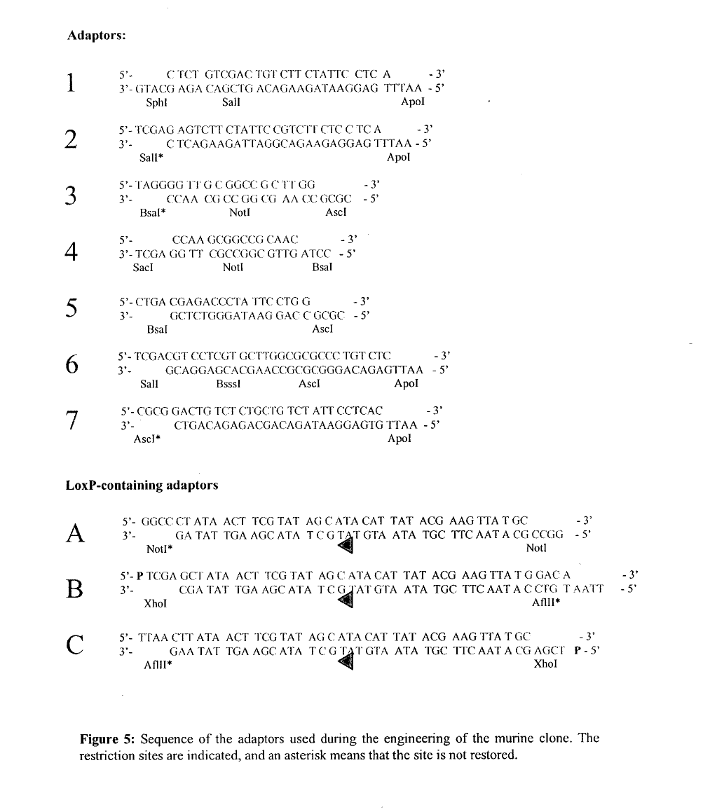

(iv) oligonucleotide adaptors containing LoxP sequences (see FIG. 5 for specific sequences).

(v) a pNEB193 cloning vector (New England Biolabs, Beverly, Mass.).

The targeting construct was completely sequenced prior to use to verify the integrity of the MSTN sequences. The regions corresponding to the 5′ and 3′ homology arms were shown by sequencing to be identical in the targeting vector (C57B 16/6N origin) and the R1 ES cell line (SV129 origin). See Nagy et al. PNAS USA 90:8424-8428 1993. A detailed description of the construction of the targeting construct follows.

Sequencing and Characterization of a Murine Genomic Lambda Clone Encompassing the Entire Myostatin Gene Plus Flanking Sequences

Two bovine and one murine commercial genomic libraries constructed in lambda replacement vectors were screened following standard procedures, using a previously described P³² labeled bovine myostatin cDNA as a probe (Grobet et al. Nature Genetics 17:71-74, 1997). The murine library was derived from the liver tissue of a male C57BL/6N mouse. Positive clones were primarily characterized using myostatin and vector-specific primers in combination with the Expand™ Long Template PCR System (Boehringer Mannheim). Cloned inserts were amplified from phage lysates using vector-specific primers and the Expand™ Long Template PCR System (Boehringer Mannheim). PCR products were sheared by sonication, and the resulting fragments were treated with Klenow enzyme. After size-selection, fragments were cloned in pUC18 and a number of clones corresponding to four equivalents of each insert were sequenced with Dye terminator cycle sequencing Ready Reaction (ABI) on an ABI373 automatic sequencer. Contig assembly was performed with the Phred/Phrap/Consed software package (Ewing et al. Genome Research 8:175-185 1998; Ewing & Green Genome Research 8:186-194 1998; Gordon et al. Genome Research 8:195-204 1998). Primers were designed to amplify the remaining gaps. PCR products derived from the phage lysates or, in the case of the bovine myostatin gene, from previously described YAC clones (Pirottin et al. Mammalian Genome 10:289-293 1999), were sequenced as previously described. The transcription unit boundaries were defined by sequencing cRACE products (Maruyama et al. Nucleic Acids Research 23:3796-3797 1995) for the 5′ end and 3′RACE products (3′ RACE system, Life Technologies) for the 3′ end. Three sets of primers lying downstream of the stop codon were used in the 3′RACE experiments. The sequences were analyzed using the Wisconsin package of the GCG group and the search for muscle-specific cis-acting response elements was performed using a logistic regression algorithm (Wasserman & Fickett Journal of Molecular Biology 278:167-181 1998).

Engineering of the Murine Clone

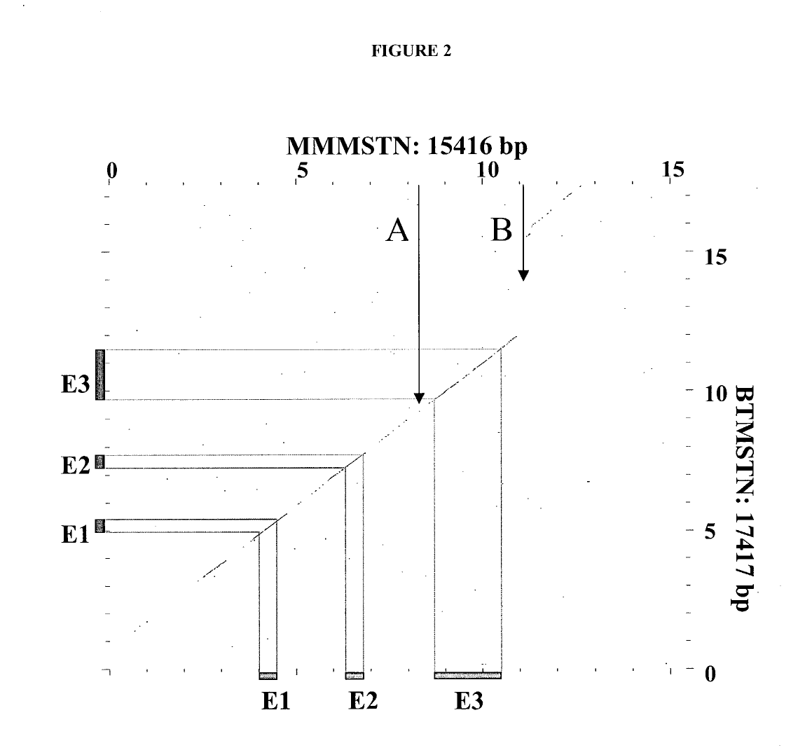

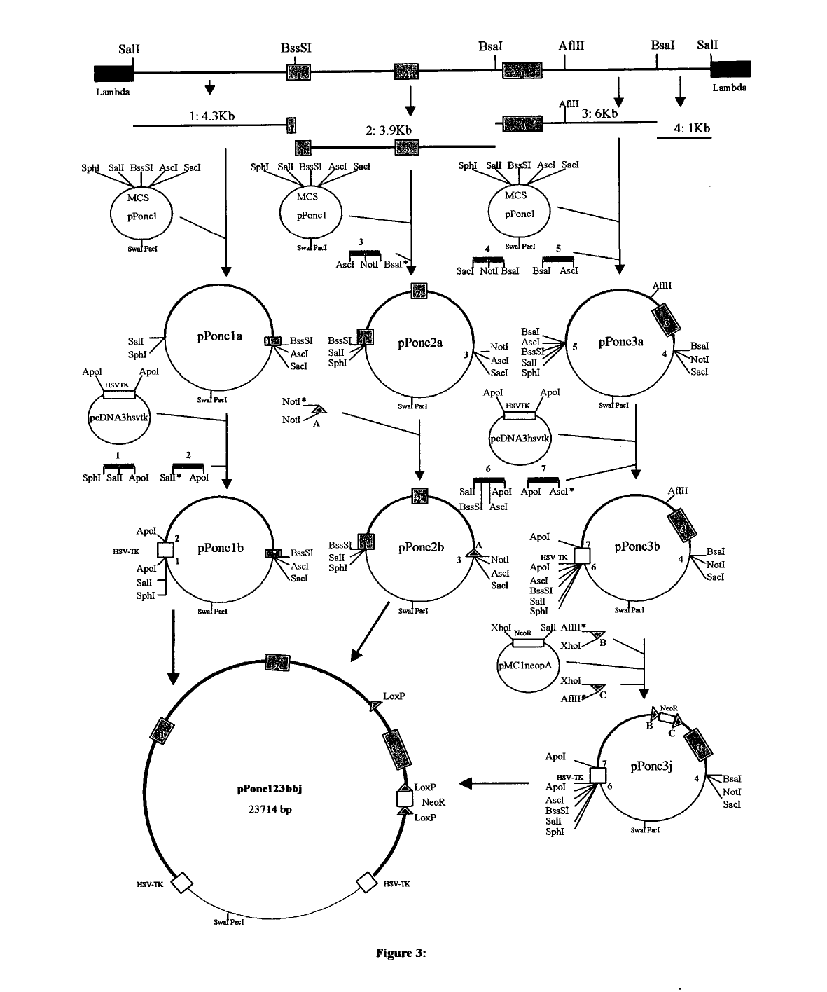

Based upon the sequence of the murine genomic clone on one hand, and on the comparison between bovine and murine sequences (Royo et al., in preparation) on the other hand, a strategy was developed to flox the third exon of the myostatin gene in a construct suitable for homologous recombination in ES cells. The target sites for introducing the exogenous sequences (LoxP sites, 34 base pairs) were chosen on each side of the third exon within regions showing poor or no homology between cattle and mouse (FIG. 2). This choice was also dependent on the availability of helpful restriction sites. FIG. 2 shows target sites for the introduction of LoxP sequences (A and B). Each dot on the graph represents a region of homology of more than 16 base pairs in a 21 base pair window. The three bovine (vertical axis) and murine (horizontal axis) exons are represented on their respective axes (E1, E2, E3). The “A” and “B” regions, indicated by arrows, are two regions of poor interspecific homology flanking the third exon, and have been targeted for the introduction of LoxP sites. The overall strategy was based on standard cloning procedures (Sambrook et al., Molecular Cloning: A Laboratory Manual. Second Edition, Cold Spring Harbor Laboratory Press, 1989) in a pUC-derived plasmid vector, including restriction endonuclease digestions and ligations and use of synthetic oligonucleotide adaptors, avoiding any PCR amplification steps (FIG. 3). Each step was monitored by specific PCR reactions and/or restriction patterns.

Modifications of a pUC-Derived Plasmid.

As shown in FIG. 3, the engineering steps require particular plasmid features. In FIG. 3, the green boxes represent the exons of the myostatin gene. The principal restriction sites are indicated for the intermediate steps. An asterisk means that the site is not restored. The red color means that the site is digested for the further step. Synthetic adaptors are indicated in blue (red for the LoxP-containing adaptors). The final construct (pPonc123bbj) is made after ligation of pPonc2b and pPonc3j inserts into linearized pPonc1b. pPonc1b, pPonc2b, and pPonc3j are made up of the respective fragments 1, 2 and 3 of the murine myostatin genomic clone modified as described hereafter and cloned in the pPonc1 plasmid which is a modification of the pNEB193 plasmid vector (New England Biolabs). Cloning was achieved using the Chameleon^{R} double-stranded site-directed mutagenesis kit (Stratagene). Five mutagenesis reactions were performed in four separate experiments on pNEB193, each targeting a different region of the plasmid: (1) at the level of the polylinker: replacement of the unique PacI site by a BssSI site; (2) in the β-Lactamase-ORI interregion, addition new PacI and SwaI sites. These sites will allow for linearization of the final construct prior to homologous recombination; (3) in the β-Lactamase coding sequence (nt 2390), removal of the first BsssI site; (4) in a non-coding region (nt 2697), removal of a second BssSI site; (5) the third BssSI site located in the ORI has not been successfully eliminated. The final modified plasmid (pPonc1) was obtained by assembling mutated restriction fragments from the modified plasmids mentioned above. Table 1 lists the primers and restriction enzymes used in the mutation-selection steps.

TABLE 1

Primers and restriction endonucleases for
site-directed mutagenesis (Chameleon^{R}).

| | |
|---|---|
| Polylinker | |
| PacI→BssSi: | P GGGCGCGCCGGATCCTCTCGTGAGTCT |
| | AGAGTCGACTG |
| Selection: | PaCI |
| Between β-Lactamase | |
| and ORI | |
| +PacI + SwaI: | P CCTTTTAAATTAAATTAATTAATTTAA |
| | ATCAATCTAAAG |
| Selection primer: | P CATCATTGGAAAACGCTCTTCGGGGCG |
| Selection: | XmnI |
| BssSI site at Nt 2697 | |
| −BssSI: | P CCTATAAAAATAGGCGTATCAGGAGGC |
| | CCTTTCGTC |
| Selection primer: | P CATCATTGGAAAACGCTCTTCGGGGCG |
| Selection: | XmnI |
| BssSI site at nt 2390 | |
| (β-Lactamase gene) | |
| −BssSI: | P GTTCGATGTAACCCACGCGTGCACCCA |
| | ACTGATC |
| Selection primer: | P CATCATTGGAAAACGCTCTTCGGGGCG |
| Selection: XmnI | |
| BssSI site at nt 1006 | |
| (ORI) | |
| −BssSI: | P CCCCTGGAAGCTCCCTCCTGCGCTCTC |
| | CTGTTCCG |
| Selection primer: | P CATCATTGGAAAACGCTCTTCGGGGCG |
| Selection: | XmnI |

The primers shown in Table 1 are numbered top through bottom, SEQ ID NO: 1; SEQ ID NO:2; SEQ ID NO:3; SEQ ID NO:4; SEQ ID NO:3; SEQ ID NO:5; SEQ ID NO:3; SEQ ID NO:6 and SEQ ID NO:3, respectively.

Cloning and Modification of the Murine Insert in pPonc1

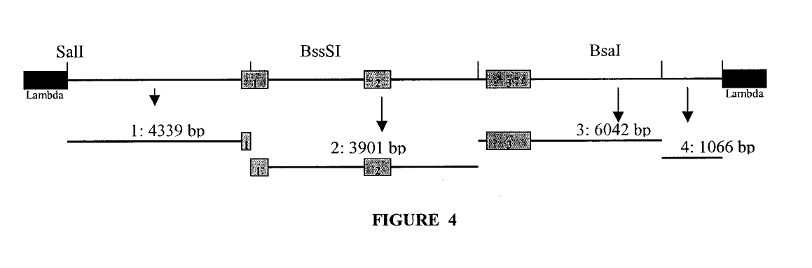

The murine genomic lambda clone has been cultured in liquid phase using standard procedures, and extracted using the Qiagen lambda midi kit. The 15.3 Kb murine insert has been excised with SalI, agarose gel isolated, and subsequently fragmented using BssSI and BsaI. The expected sizes are shown in FIG. 4. Hereafter the 5′ to 3′ ends of the fragments are referred to for the exon1 to exon 3 polarity. After agarose gel isolation, the 3′ 1066bp BsaI-SalI fragment was discarded, while the three other were separately cloned and manipulated in pPonc1.

Fragment 1: SalI-BsssI, 4339 bp

Cloning of fragment1 in pPonc1: The linearized form of a partially BssSI-digested pPonc1 was digested to completion with SalI. After agarose gel fractionation, the 2.7 Kb band was isolated and ligated to the murine fragment 1, giving rise to the pPonc1a clone.

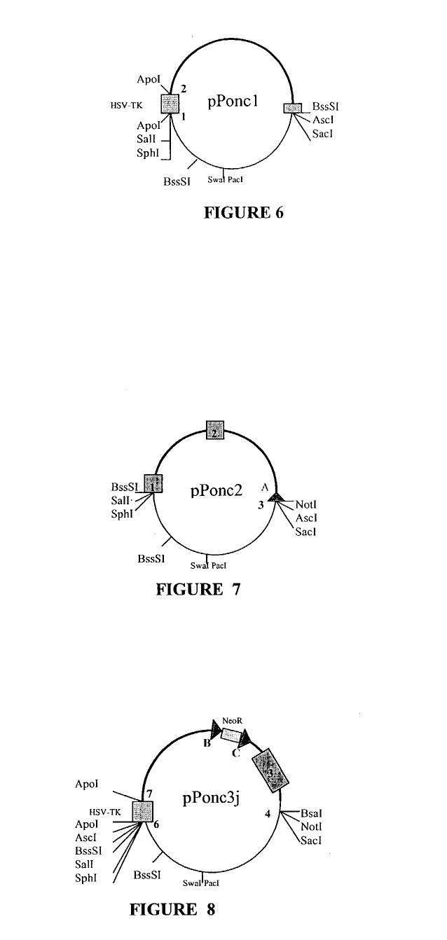

Addition of an HSV-thymidine kinase eukaryotic expression cassette to the 5′end of fragment I: An HSV-thymidine kinase eukaryotic expression cassette, contained in a 2676 base pair ApoI-ApoI fragment, was isolated from the pcDNA3hsvtk plasmid (gift of F. Princen, ULg). After digestion of pPonc1 a with SphI and SalI, two adaptors (1 and 2, FIG. 5; it is noted in FIG. 5 the restriction sites are indicated and an asterisk means that the site is not restored) and the 2676 base pair ApoI fragment were ligated to the digested plasmid. The resulting clone has been called pPonc1b and is shown in FIG. 6. (In FIG. 6, the thick line represents the murine insert. The principal useful restriction sites are shown. The square box represents the HSV-TK expression cassette and the rectangle box represents the beginning of the murine myostatin first exon.)

Fragment II: BssSI-BsaI, 3901 bp

Cloning of fragment II in pPonc1: The linearized form of a partially BssSI-digested pPonc1 was digested to completion with AscI. After agarose gel fractionation, the 2.7 Kb band was isolated and ligated to the murine fragment 2 using one adaptor (3, FIG. 5), resulting in the pPonc2a clone. Addition of a LoxP site at the 3′ end of the genomic insert: An adaptor (A, FIG. 5) including a LoxP site was ligated to the NotI linearized pPonc2a plasmid. Due to the polar nature of the LoxP sequence, two different forms were generated. The one characterized by a 3′ to 5′ orientated (as arbitrarily fixed) LoxP and by a restored NotI site at the 3′ end of the insert was chosen for further steps (pPonc 2b, FIG. 7; in FIG. 7, the thick line represents the murine insert. The principal useful restriction sites are shown. The two numbered boxes represent the end of the first and the complete second exon of the murine myostatin gene. The arrow represents the “A” LoxP site and the number 3 refers to a synthetic adaptor (see FIG. 5 for adaptors sequences).

Fragment III: BsaI-BsaI, 6042 base pairs.

Cloning of fragment III in pPonc 1: pPonc 1 was digested to completion with AscI and SacI. The linear plasmid was ligated to the fragment 2 by means of two adaptors (4 and 5, FIG. 5), generating the pPonc3a clone.

Addition of an HSV-thymidine kinase expression cassette to the 3′end of fragment III: The previously described 2676 base pair ApoI-ApoI fragment (containing the HSV-thymidine kinase expression cassette) was ligated to the AscI and SalI digested pPonc3a plasmid by means of two adaptors (6 and 7, FIG. 5). The resulting construct (pPonc3b) is characterized by the presence of an HSV-TK expression cassette located at the 3′ end of the genomic insert. Insertion of a floxed neomycin phosphotransferase eukaryotic expression cassette at the 3′ side of exon3: The neomycin resistance cassette is contained in a 1146 base pair SalI-XhoI fragment of the pMC1neoPolyA vector (Stratagene). Two LoxP-containing adaptors (B and C, FIG. 5) were ligated to each other by their phosphorylated XhoI extremities and to the AfIII-linearized pPonc3b. Out of the two generated forms, the one characterized by two LoxP sites tandemly repeated in a 3′ to 5′ orientation was chosen for further manipulations (pPonc 3c). The 1146 base pair neo (SalI-XhoI) fragment was ligated to XhoI linearized pPonc3c, resulting in its insertion between the two LoxP sites (pPonc3j, FIG. 8, in FIG. 8, the thick line represents the murine insert. The useful restriction sites are shown. The square box represents the HSV-TK expression cassette and the rectangle box represents the third exon of the murine myostatin gene. The arrows represent the “B” and “C” LoxP site and the numbers (4, 7 and 6) refer to synthetic adaptors; see FIG. 5 for adaptors sequences).

Assembly of the Three Modified Fragments

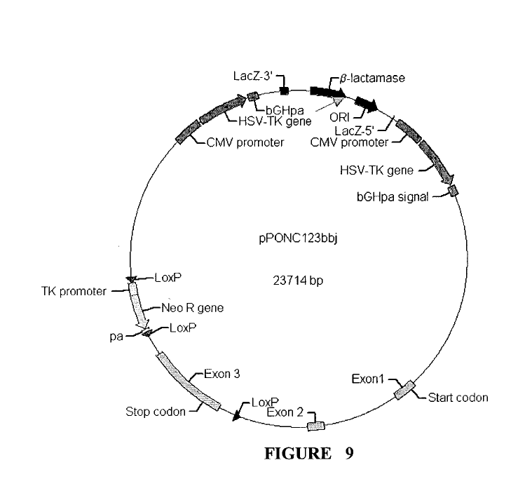

The final assembly was made up through a two steps procedure. The second and third modified murine fragments were sequentially inserted into the linearized pPonc1b clone. The pPonc1b plasmid was partially digested with BssSI and the linearized form (+/−9.7 Kb) was subsequently digested to completion with AscI. The 9.7 Kb fragment, corresponding to the linearized form, was kept as the acceptor plasmid for the second fragment. The pPonc2b plasmid was digested with BssSI and AscI, and the 3.9 Kb (insert) fragment was ligated to the linearized pPonc1b, generating the pPonc12bb clone. The latter has been digested with AscI and NotI, and ligated to the 10 Kb insert generated by the AscI and NotI digestions of pPonc3j. The resulting final clone (pPonc123bbj) is represented in FIG. 9. In FIG. 9, the light-colored arrow (below the β-lactamase cassette) points towards unique PacI and Swal sites that are used for further linearization of the plasmid prior to electroporation.

Sequencing and Testing the Eukaryotic Expression Cassettes

In order to check the complete sequence of the final construct and to monitor efficiency of the positive (neoR) and negative (HSV-TK) selection genes, both complete sequencing and in vitro testing of the construct were undertaken.

Sequencing of pPonc123bbj

A maxi-preparation of the pPonc123bbj plasmid was made using an endotoxin-free plasmid maxi-kit (Qiagen). The whole insert, except the HSV-TK expression cassettes, was sequenced directly from the plasmid. Both HSV-TK cassettes were amplified using the Expand™ Long Template PCR System (Boehringer Mannheim) with the primer reported in bold characters in Table 2. The plasmid and the PCR products were sequenced by primer walking using the primers reported in Table 2 using a BigDye™ terminator cycle sequencing Ready Reaction kit (ABI) on an ABI377 automatic sequencer. Contig assembly was performed with the Phred/Phrap/Consed software package (Ewing et al. Genome Research 8:175-185 1998; Ewing & Green Genome Research 8:186-194, 1998; Gordon et al. Genome Research 8:195-204, 1998).

TABLE 2

Primers used for sequencing the construct.
The primers written in bold were used for PCR
amplification of the HSV-TK expression cassettes
which were sequenced using the “tk1” to
“tk7” primers.

| | | |
|---|---|---|
| rev25 | CACACAGGAAACAGCTATGACC | (SEQ ID NO:27) |
| | ATGA | |
| tk1 | ACTGCCCACTTGGCAGTACATC | (SEQ ID NO:28) |
| | AAG | |
| tk2 | TAACTAGAGAACCCACTGCTTA | (SEQ ID NO:29) |
| | CTG | |
| tk3 | TTCCGAGACAATCGCGAACATC | (SEQ ID NO:30) |
| | TAC | |
| tk4 | CGAGCGGCTTGACCTGGCTATG | (SEQ ID NO:31) |
| | CTG | |
| tk5 | CACCCCAGGCTCCATACCGACG | (SEQ ID NO:32) |
| | ATC | |
| tk6 | TGCGGTGGGCTCTATGGCTTCT | (SEQ ID NO:33) |
| | GAG | |
| tk7 | CTTGTTCCAAACTGGAACAACA | (SEQ ID NO:34) |
| | CTC | |
| 3a | TCGAGAGTCTTCTATTCCGTCT | (SEQ ID NO:35) |
| | TCTCCTCA | |
| 24up | GAGGAGGTATGAATGTCATTTC | (SEQ ID NO:36) |
| | AAC | |
| mm30 | TATTCCTTTCATACCCTAACTC | (SEQ ID NO:37) |
| | AAC | |
| mm31 | AGCTGATTATCCATGCTTTTCA | (SEQ ID NO:38) |
| | TAG | |
| mm55 | CATTAAAGTTCTTGCAGTGTAG | (SEQ ID NO:39) |
| | TAG | |
| 24dn | AGTGGAAGAAATTCTCTCTTCA | (SEQ ID NO:40) |
| | CTC | |
| mm35 | CTCGACAGCACAGAATTCATGA | (SEQ ID NO:41) |
| | ATG | |
| mm36 | GTAGCTCACCTCACCCTGCATG | (SEQ ID NO:42) |
| | TTC | |
| mm37 | ACAACCATATTTTTAGAATGCT | (SEQ ID NO:43) |
| | GTG | |
| 13up | TCAGCTCTGACTTTATGAACAA | (SEQ ID NO:44) |
| | ATG | |
| mm34 | CCAGCTACCCAGATTCCCCACT | (SEQ ID NO:45) |
| | GAG | |
| mm45 | AAAGAGCAAGCCCTTCTGCTTC | (SEQ ID NO:46) |
| | AAG | |
| mm46 | GCAATATAAGTAGCTAAATGTA | (SEQ ID NO:47) |
| | GTC | |
| 13dn | AAGAGGGCCAGATCACCTCAGG | (SEQ ID NO:48) |
| | GTG | |
| mm39 | TATTAGAGCAGGCCTATAAAGT | (SEQ ID NO:49) |
| | CAG | |
| 22bup1 | TTTGTTCAGCTCTTTAAGAGTT | (SEQ ID NO:50) |
| | CAC | |
| mm52 | CTCCTGTTTGGGAAGCTGAGGA | (SEQ ID NO:51) |
| | GTC | |
| 22bup2 | TGACAGTAAAGTGCAATCTGTG | (SEQ ID NO:52) |
| | TTC | |
| mm40 | TTATCTACTCGGCCTAAGTACA | (SEQ ID NO:53) |
| | GAG | |
| mm56 | TGCGTTAAGTGCTGGGTAATTA | (SEQ ID NO:54) |
| | GAG | |
| 22bdn | CAAGAGTTTTACAGAGATTAAT | (SEQ ID NO:55) |
| | AAG | |
| 10up1 | TAAAACCCTGTCTGTCACAAGT | (SEQ ID NO:56) |
| | CAC | |
| gdf8-17 | CGGACGGTACATGCACTAATAT | (SEQ ID NO:57) |
| | TTCAC | |
| souprimex | ATCATTTTAAAAATCAGCACAA | (SEQ ID NO:58) |
| | TC | |
| sou-nest3′ | GCTGCGCCTGGAAACAGCTCCT | (SEQ ID NO:59) |
| | AAC | |
| 1stsounew | TCACTGCTGTCATCCCTCTGGA | (SEQ ID NO:60) |
| | CGTCG | |
| 10dn1 | TATATCTGTTAAAGTATATCAA | (SEQ ID NO:61) |
| | CAG | |
| 16dn | ATTTCATTGTCGGTATGTTTCT | (SEQ ID NO:62) |
| | CAG | |
| mm57 | CTATAATGTAAGGACTGTGAGA | (SEQ ID NO:63) |
| | TTC | |
| 16up | TATTAAATGCATTATCATGAGC | (SEQ ID NO:64) |
| | CAC | |
| 26and | ACAGAAATCTTTCGTGTTCTGC | (SEQ ID NO:65) |
| | CTG | |
| mm58 | ACATTTCAGGCAGTTCCTGTTT | (SEQ ID NO:66) |
| | GAG | |
| mm59 | GGAAAAGCAATTGTTAGTGCTG | (SEQ ID NO:67) |
| | AAC | |
| i1-seq7-5′ | CTCCAGACTGACTGGTACAGCT | (SEQ ID NO:68) |
| | GCTC | |
| mm60 | TTCTGAACTATGAATGAAGTTC | (SEQ ID NO:69) |
| | CAC | |
| gdf8-11 | ACAGTGTTTGTGCAAATCCTGA | (SEQ ID NO:70) |
| | GAC | |
| gdf8-12 | CAATGCCTAAGTTGGATTCAGG | (SEQ ID NO:71) |
| | CTG | |
| mm61 | ATAAGCCAGACAAAGTATCTTA | (SEQ ID NO:72) |
| | CTC | |
| 26aup | TGAAAAATGTTGGTTCACATAA | (SEQ ID NO:73) |
| | AG | |
| 26dn | CTATATACATATCATGGCTTCA | (SEQ ID NO:74) |
| | AC | |
| mm62 | TAGTGAGTCAGTGATAGGACAA | (SEQ ID NO:75) |
| | GAC | |
| 26up | ATTGAACTTGGGAATATACAGT | (SEQ ID NO:76) |
| | CTG | |
| 18up | AAGGAATATCACACTAACCACC | (SEQ ID NO:77) |
| | TTG | |
| mm63 | GTGGTTAGTGTGATATTCCTTA | (SEQ ID NO:78) |
| | GAG | |
| mm64 | TATACATACAGCCACTGTCATC | (SEQ ID NO:79) |
| | ATG | |
| 18dn | TGCTATTATGTCTGATAATAGT | (SEQ ID NO:80) |
| | ATG | |
| bt32 | TCCCGGAGAGACTTTGGGCTT | (SEQ ID NO:81) |
| 12up | TGGGTGTGTCTGTCACCTTGAC | (SEQ ID NO:82) |
| | TTC | |
| gdf8-14 | CCCCCTCACGGTCGATTTTGAA | (SEQ ID NO:83) |
| | GCC | |
| gdf8-15 | TCCCATCCAAAGGCTTCAAAAT | (SEQ ID NO:84) |
| | C | |
| mm50 | CCCATTAATATGCTATATTTTA | (SEQ ID NO:85) |
| | ATG | |
| gdf8-13 | GAGCACCCACAGCGGTCTACTA | (SEQ ID NO:86) |
| | CCAT | |
| mm54 | GTAGACCGCTGTGGGTGCTCAT | (SEQ ID NO:87) |
| | GAG | |
| mm42 | TGGTCTGCTGAGTTAGGAGGGT | (SEQ ID NO:88) |
| | ATG | |
| mm47 | TACAAAGGCTACATATAGATTC | (SEQ ID NO:89) |
| | TTC | |
| mm49 | CGGAAGAATCTATATGTAGCCT | (SEQ ID NO:90) |
| | TTG | |
| mm53 | GCACAGCGGGAGTGACTGCTGC | (SEQ ID NO:91) |
| | ATC | |
| sou3′5′1 | AATGTATTGTACTCATAGCTAA | (SEQ ID NO:92) |
| | ATG | |
| mm48 | AATAATTTCATTTAGCTATGAG | (SEQ ID NO:93) |
| | TAC | |
| mmrace3′ | CATGGTGGCTGTATCTATGAAT | (SEQ ID NO:94) |
| | GTG | |
| mm65 | AATTGGCAGTGGTATATACTCC | (SEQ ID NO:95) |
| | TAG | |
| 12dn | CTACCTTCATCAGGTCAGGGAT | (SEQ ID NO:96) |
| | GTG | |
| neocas1 | GGGCTGACCGCTTCCTCGTGCT | (SEQ ID NO:97) |
| | TTAC | |
| mm66 | CATCGCCATGGGTCACGACGAG | (SEQ ID NO:98) |
| | ATC | |
| mm67 | GGGCACCGGACAGGTCGGTCTT | (SEQ ID NO:99) |
| | GAC | |
| neocas2 | CATTCCAGGCCTGGGTGGAGAG | (SEQ ID NO:100) |
| | GCT | |
| mm68 | AATTGTGACATGATAAAAATCC | (SEQ ID NO:101) |
| | ATC | |
| 17up1 | TTTTGATGGATTTTTATCATGT | (SEQ ID NO:102) |
| | CAC | |
| 17up2 | TGTGTCTTAGACCTCAATGGCC | (SEQ ID NO:103) |
| | ATG | |
| mm69 | GTACATTAGAATGGATGGTTTG | (SEQ ID NO:104) |
| | CAG | |
| 17dn | TTTGTTGTTCTCAGATTTCTGT | (SEQ ID NO:105) |
| | GGC | |
| mm70 | CTAACCACTCCAAATCACTCTG | (SEQ ID NO:106) |
| | TTC | |
| 22aup | CTTAATGTCCCTGGGAGCAGAT | (SEQ ID NO:107) |
| | CTG | |
| mm44 | TCAGTCCCTGACAATACAGTCA | (SEQ ID NO:108) |
| | CTG | |
| 22adn | GTCAGGTGTGGTAGCCTAGAAA | (SEQ ID NO:109) |
| | TGC | |
| mm71 | TTTGCTTTGATGATAGTGAAGC | (SEQ ID NO:110) |
| | GTC | |
| mm72 | GGAGTGAACAAACACTGAGTTC | (SEQ ID NO:111) |
| | CAG | |
| mm73 | TAAACTGCCCATAGACAGTGTA | (SEQ ID NO:112) |
| | TTG | |
| mm74 | CATCCAGCTCAGCCTATGTGTT | (SEQ ID NO:113) |
| | GAG | |
| 14bup | TGTAAGGATGATTAGAAATGAC | (SEQ ID NO:114) |
| AAC | | |
| 17a | CGCGGACTGTCTCTGCTGTCTA | (SEQ ID NO:115) |
| | TTCCTCAC | |
| 18a | AATTGTGAGGAATAGACAGCAG | (SEQ ID NO:116) |
| | AGACAGTC | |
| 19a | TCGACGTCCTCGTGCTTGGCGC | (SEQ ID NO:117) |
| | GCCCTGTCTC | |
| seq24 | CGACGTTGTAAAACGACGGCCA | (SEQ ID NO:118) |
| | GT | |

Testing the Eukaryotic Expression Cassettes.

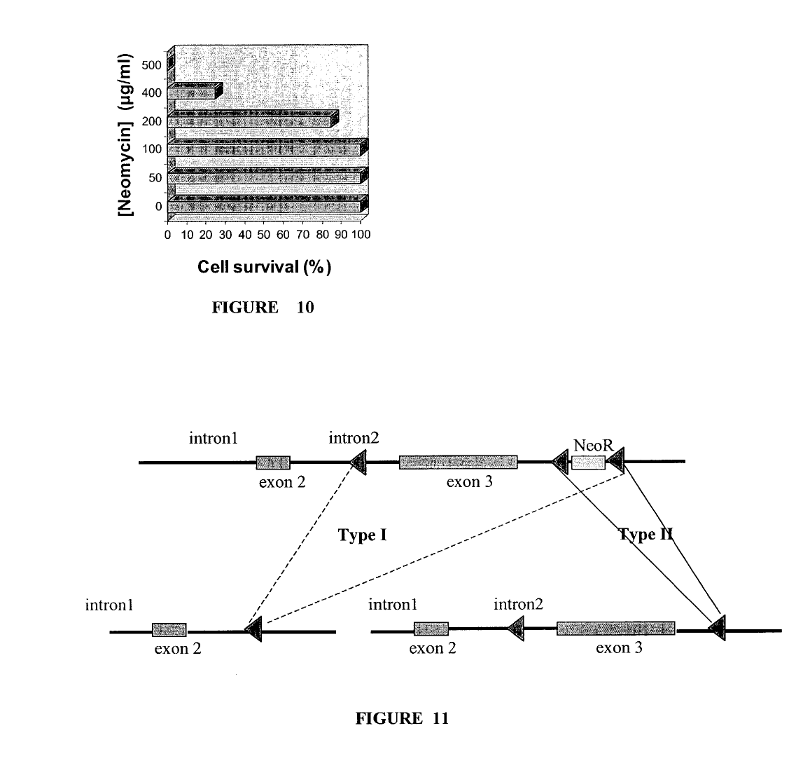

In order to test the potency of the pPONC123bbj construct to confer neomycin resistance, as well as gancylovir susceptibility, several CHO-K1 cell lines were established that contain this construct in a stably integrated form. Determination of the selection conditions: Appropriate antibiotic selection conditions for the parental cell line were first determined. A confluent CHO-K1 cells 175 cm² dish was split 1:15 into HAM F12 Kaighn's modification medium supplemented with 10% fetal bovine serum and containing various concentrations of neomycin (0, 50, 100, 200, 400 and 500 μg/ml). Cells were incubated for days, fed with selective medium every 3 days, and examined every day for cell viability (FIG. 10).

Establishment of the neomycin resistant CHO-bbj cell lines: In a six-well plate, approximately 1 to 3.10⁵ CHO-K1 cells were seeded per well in 2 ml of the appropriate growth medium supplemented with serum. Cells were incubated at 37° C. in a CO₂ incubator until the cells were 50 to 70% confluent. The following solutions were prepared in 12×75 mm sterile tubes: Solution A: 2 μg of plasmid DNA diluted into 120 μl serum-free medium Optimem™ I Reduced Serum Medium (Life Technologies). Solution B: 19 μl of Lipofectamine™ Reagent (Life Technologies) into 100 μl serum-free medium. The two solutions were combined, mixed gently, and incubated at room temperature for 45 min. Meanwhile, cells were washed once with 2 ml of HAM F12 serum-free medium and 0.8 ml of HAM F12 serum-free medium was added to each well. The DNA-lipid complexes were overlayed the onto the washed cells and incubated for 6h at 37° C. in a 5% CO₂ incubator. After this incubation, 1 ml of growth medium containing twice the normal concentration of serum was added without removing the transfection mixture. Medium was replaced at 24 h following start of transfection. At 72 h posttranfection, cells were passaged 1:10 into the selective medium containing 500 μg/ml of neomycin. Three large, healthy colonies were picked. These CHO-bbj neomycin resistant cell clones were propagated and passaged onto 175 cm² plates in selective medium every 3 days.

Functional testing of the HSV-thymidine kinase cassette: First, the susceptibilty of the parental CHO-K1 cell line to gancyclovir was determined (Cymeven-Gancyclovirum, Roche). Diluted CHO-K1 cells incubated with increasing concentrations of the drug indicated that they resist up to 100 μM. Similar experiments were performed on the CHO-bbj stable cell lines. Out of these three cell lines, only one of them showed significant level of cell death using 100 μM of gancyclovir. In conclusion, these results indicate that the neomycin resistance gene and, at least, one of the two HSV-TK expression cassettes are functional in this cell system.

The second stage in the preparation of the mouse of the instant invention is carrying out gene targeting by homologous recombination using the construct described above in ES cells. Gene targeting was performed in R1 cells (Nagy et al. PNAS USA 90:8424-8428 1993) using standard procedures (Torres & Kuhn Laboratory Protocols for Conditional Gene Targeting. Oxford University Press, New York 1997). The targeting vector was linearized with PacI and 25 ug of the resulting product was used to electroporate 10⁷ R1 RS cells. Positive-negative selection was performed using G418 (300 ug/ml) and gancyclovir (2 uM). Resistant clones were picked in triplicate in 96-well plates (one copy was used for freezing and two were used for DNA extraction). Screening for the expected targeting event was performed by PCR and confirmed by Southern blotting. Positive clones were expanded and 10⁷ cells transfected with 5 ug of supercoiled pMC-cre plasmid (Gu et al. Cell 73:1155-1164 1993) in order to delete the neomycin resistance cassette and obtain a MSTN^{flox} allele (as shown in FIG. 12). The resulting clones were plated at low density and subsequently picked in triplicate in 96-well plates (one copy for freezing, one copy for monitoring the G418 sensitivity, and one copy for DNA extraction). G418 sensitive clones were analyzed by PCR to identify those having undergone the deletion event characterizing the MSTN^{flox} alleles.

The third stage of the instant invention is the generation of the transgenic mouse. It is noted that the MCK-Cre mice (these mice express the cre recombinase under the control of the murine muscle creatine kinase promoter) used in these experiments were provided by Dr. C. Ronald Kahn at the Joslin Diabetes Center in Boston, Mass.

2.5 day-old CD1 morulae were harvested and aggregated with targeted ES cells as described (Torres & Kuhn Laboratory Protocols for Conditional Gene Targeting. Oxford University Press, New York 1997). Uterine transfer was performed the next day in C57BL×CBA psuedopregnant mothers using standard procedures (Hogan et al. Manipulating the Mouse Embryo, a laboratory manual, second edition, Cold Spring Harbor Laboratory Press, 1994). Resulting male chimeras exhibiting a patched coat color were mated to CD1 females. One of the male chimeras transmitted the R1 genome to all its 13 offspring as judged from their coat color. As expected, half of these (three males and four females) also inherited the floxed myostatin allele (genotype: MSTN^{+flox}). Offspring inheriting the MSTN^{flox} allele were positively identified by PCR using DNA extracted from the tail tip of colored individuals. These mice were then intercrossed to produce a homozygous MSTN^{flox/flox} line. Next in order to produce mice harboring a constitutive deletion of the third MSTN exon, an MSTN^{flox/flox} male was mated to an FVB female. The resulting zygotes were harvested using standard procedures and microinjected with 1 ng/ul of supercoiled pCAGGS-cre plasmid in which the expression of cre is dependent on the chicken beta actin promoter (Araki et al. PNAS USA 92:160-164 1995). The resulting offspring were screened by PCR for the cre-mediated deletion of the third exon. Out of the 17 offspring, 4 received the non-recombined MSTN^{flox} allele while 8 inherited a recombined MSTN^{Δ} allele. Two of these were intercrossed to generate MSTN^{Δ/Δ} descendents.

When carrying out methods to generate transgenic animals it is important to monitor the genetic events as a control. To monitor the in vivo cre-mediated excision of exon 111, a multiplex PCR assay was developed amplifying a 353 base pair control fragment located −3 Kb upstream of the MSTN transcription start site (UP-primer: 5′ AGTGGAAGAAATTCTCTCTTCACTC-3′; SEQ ID NO:40; DN-primer: 5′-GTAGCTCACCTCACCCTGCATGTTC-3′; SEQ ID NO:42), as well as a 195 base pair fragment specific for the deleted MSTN^{flox} allele (UP-primer:5′CCATATAGTGCTCAGAAAGAGCTAC-3′; SEQ ID NO:119; (5′ TGGGCTAATTATGAATTATTCACTC-3′; SEQ ID NO: 120). The approximate positions of the corresponding primer pairs are shown in FIG. 12.

MSTN expression was monitored by RT-PCR amplification of a 397 base pair fragment, using primers located respectively in exons II (5′-AGACTCCTACAACAGTGTTTGT-3′; SEQ ID NO:121) and III (5′-TCCCATCCAAAGGCTTCAAAATC-3′; SEQ ID NO:84). As a positive control, a β-actin RT-PCR product of 698 base pairs was simultaneously amplified with primers located respectively in exons IV (5′-ACCTTCAACACCCCAGCCATGTACG-3′; SEQ ID NO:122) and VI (5′CTGATCCACATCTGCTGGAAGGTGG-3;′ SEQ NO:123) (Wu et al. Cardiovascular Research 45:994-1000 2000). Total RNA was extracted from heart, liver, spleen, peritoneal fat, gastrocnemius plantaris muscle and pectoralis muscle using Trizol® (Invitrogen, Carlsbad, Calif.). First strand cDNA synthesis was carried out in a reaction volume of 20 ul starting from 2 ug total RNA per sample, using an oligo(dT)₁₆ as primer and PowerScriptT™ reverse transcriptase (BD Biosciences/Clontech, Palo Alto, Calif.).

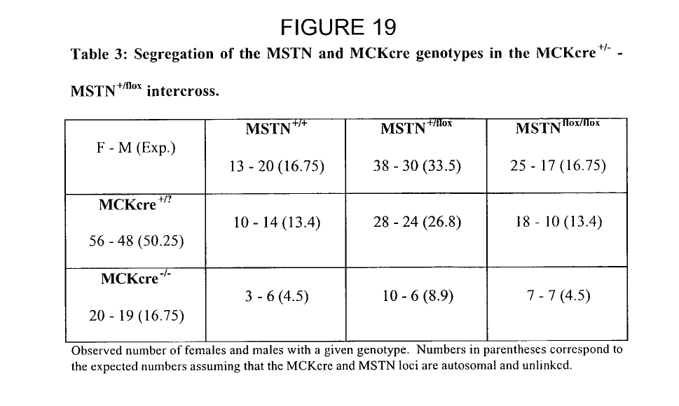

Subsequently, MSTN^{flox/flox} females were mated to transgenic males expressing cre-recombinase under the dependence of murine muscle creatine kinase (MCK) promoter (Brining et al. Molecular Cell 2:559-569 1998). The resulting MCKcre^{+/-} MSTN^{+/flox} mice were intercrossed to generate an F2 population of 134 individuals. Their MCKcre and MSTN genotypes were determined by PCR, revealing the segregation ratios expected for a dihybrid cross. (See FIG. 19, p=0.65).

TABLE 3
(FIG. 19): Segregation of the MSTN and MCKcre
genotypes in the MCKcre^{+/-}
MSTN^{+/flox} intercross

| | MSTN^{+/+} | MSTN^{+/flox} | MSTN^{flox/flox} |
|---|---|---|---|
| F-M (Exp.) | 13-20(16.75) | 38-30(33.5) | 25-17(16.75) |
| MCKcre^{+/?} | 10-14(13.4) | 28-24(26.8) | 18-10(13.4) |
| 56-48 (50.25) | | | |
| MCKcre^{-/-} | 3-6(4.5) | 10-6(8.9) | 7-7(4.5) |
| 20-19 (16.75) | | | |

Table 3 (FIG. 19) shows the observed number of females and males with a given genotype. The numbers in parentheses correspond to the expected numbers assuming that the MCKcre and MSTN loci are autosomal and unlinked.

All of the F2 generation mice were weighted at two, three, four and five months of age. 81 randomly selected mice were sacrificed at 5 months and dissected. Tissue samples were collected from 8 F2 mice (two MCKcre^{+/?} MSTN^{+/+}, three MCKcre^{+/?} MSTN^{+/flox}, three MCKcre^{+/?} MSTN^{flox/flox}, all males) for morphometric and histological analyses. The weight of the carcass (skinned body minus all internal organs and associated fat and connective tissue), “shoulder weight” (skinned left forelimb cut at wrist-level), “leg weight” (skinned left leg cut at knee level), weight of the dissected pectoralis muscles and weight of individual organs (heart, lungs, kidneys, liver, spleen) were determined.

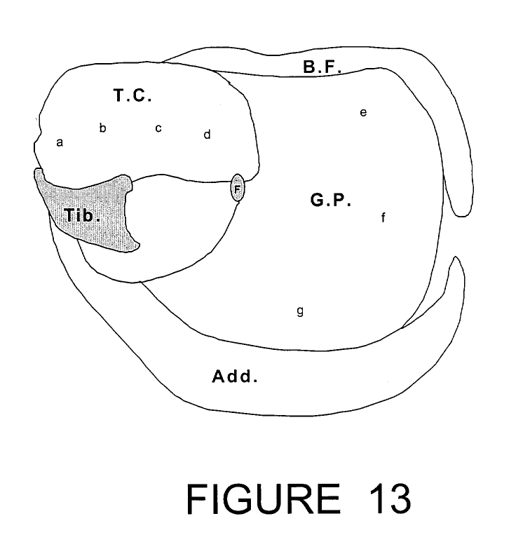

Legs of the five month old mice were stretched on a solid support and fixed in 4% buffered formaldehyde following standard procedures. After fixation, a 5 mm-wide transversal slice centered on the widest part of the lower leg was treated in EDTA saturated phosphate buffered saline for one week. After paraffin embedding, transversal sections taken from the center of the slices were stained with hematoxylin-eosin. Sections were photographed with Nomarski optics and a Leica digital camera. Image processing was realized using the analySIS^{R}3.0 image and analysis software (Soft Imaging System GmbH, Münster, Germany). “Total leg” area, area of the “tibialis cranialis” muscle group (tibialis cranialis, extensor digitorum longus and lateralis, peroneus longus), area of the “gastrocnemius plantaris” muscle group (gastrocnemius caput laterale and mediale, soleus, flexor digitorum longus), area of the “biceps femoris” and area of the “adductor” muscle group (adductor, gracilis, semimembranosus and semitendinosus) defined as illustrated in FIG. 13 were measured for each animal. In FIG. 13, the muscle groups considered in these experiments are the “tibialis cranialis” group (T.C.), the “gastrocnemius plantaris” group (G.P.), the “adductor” and the “biceps femoris”. Approximate locations of the areas in which individual myofiber area was recorded are indicated: a) tibialis cranialis, b) extensor digitorum longus, c) extensor digitorum lateralis, d) peroneus longus, e) gastrocnemius caput lateralis, f) flexor digitorum longus and g) gastrocnemius caput mediale.

A microscopic field was photographed at 20× magnification for each of the four muscles in the “tibialis cranialis” group and for three muscles in the “gastrocnemius plantaris” group (gastrocnemius caput laterale and mediale, flexor digitorum longus) (FIG. 13). A grid defining 20 identical squares was superimposed on each image and the area occupied by the myofibre spanning the center of each of the 20 squares was measured. This yielded 20×4=80 measurements per animal for the tibialis cranialis group and 20×3=60 measurements per animal for the gastrocnemius plantaris 20 group.

Phenotypic data were analyzed using a linear model including an overall mean, a fixed effect corresponding to sex (male, female), a fixed effect corresponding to genotype (MCKcre^{-/-} MSTN^{+/+}, MCKcre^{-/-} MSTN^{+/flox}, MCKcre^{-/-} MSTN^{flox/flox}, MCKcre^{+/?} MSTN^{+/+}, MCKcre^{+/?} MSTN^{+/flox} and MCKcre^{+/?} MSTN^{flox/flox}) and an error term. Statistical analyses were performed using the GLM procedure of the SAS package (SAS Institute Inc., Cary, NC).

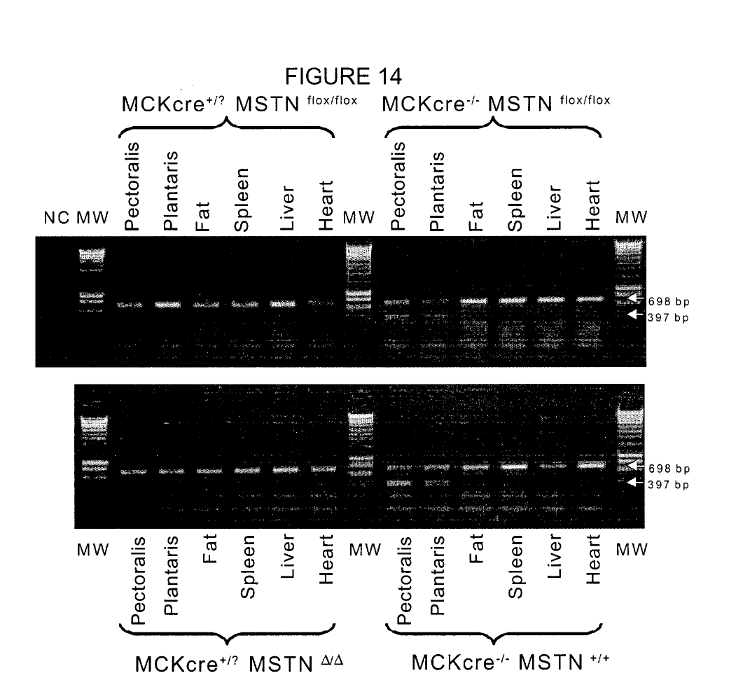

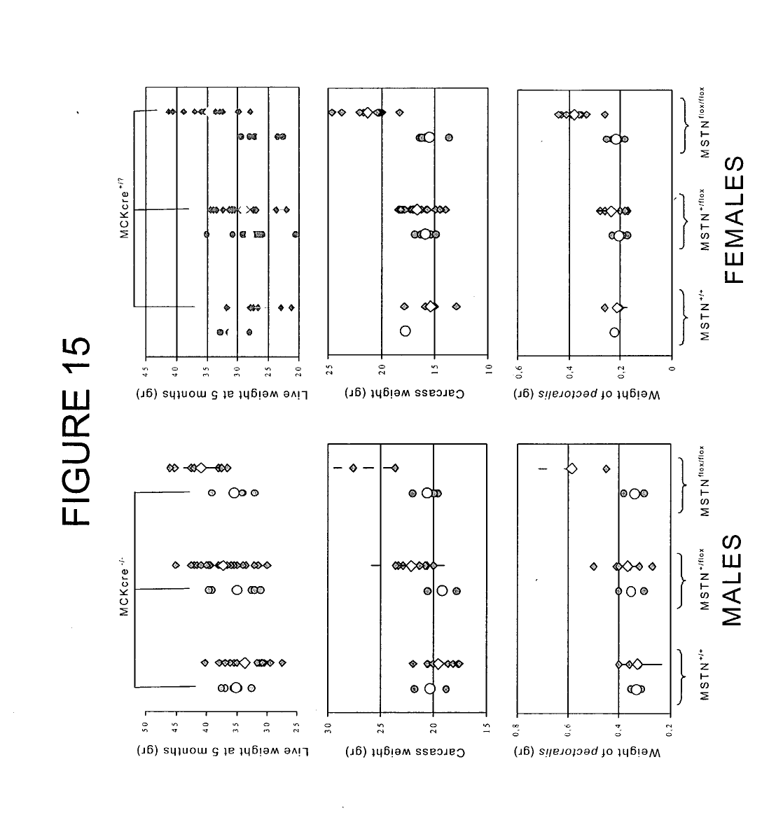

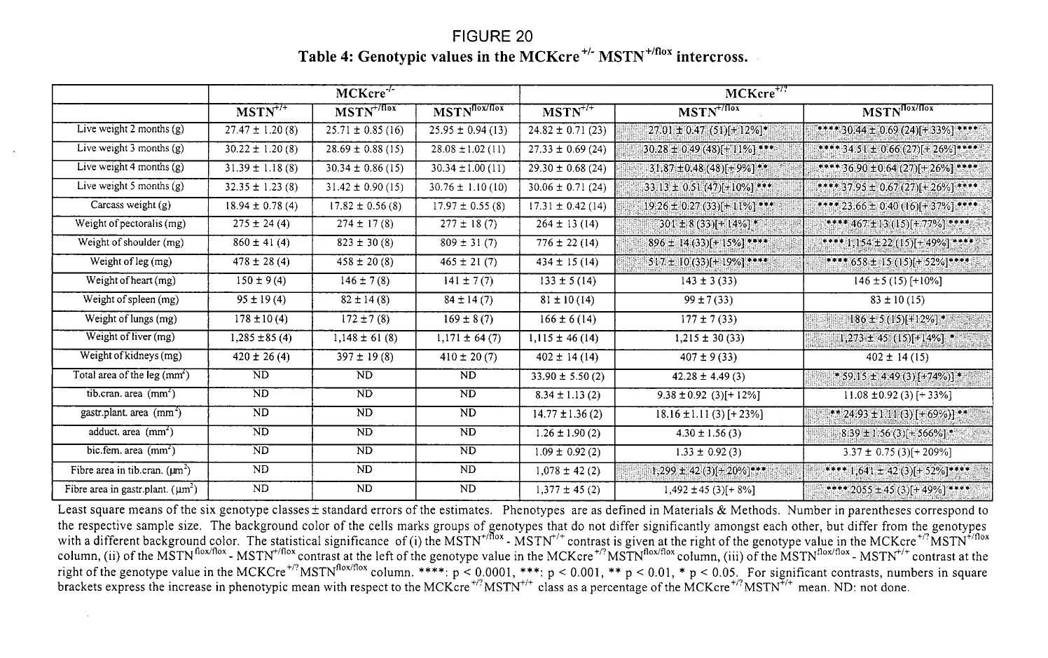

Next it was verified whether the insertion of the loxP sites on either side of exon III might interfere with the functionality of the MSTN gene. MSTN mRNA levels were compared between MCKcre^{-/-} MSTN^{+/+} and MCKcre^{-/-} MSTN^{flox/flox} in a range of tissues of two-month old mice (FIG. 14). In FIG. 14, the 397 base pair fragment is specific for MSTN transcripts containing the IInd and IIIrd MSTN exons. The 698 base pair product serves as an internal control for the assay and is specific for β-actin transcripts containing the IVth and VIth β-actin exons. “MW” and “NC” stand for molecular weight marker and negative control, respectively. In both genotypes, MSTN specific RT-PCR product was detected in skeletal muscle (pectoralis and gastrocnemius plantaris), but not in heart, liver, spleen and abdominal fat. The intensity ratios between the bands corresponding to MSTN and the β-actin control were comparable in both genotypes. These results indicate that the lox P sites did not affect the tissue-specific expression pattern of MSTN nor its expression level in skeletal muscle. A possible effect of loxP sites was further assessed by comparing live weight, carcass weight and weight of individual organs from MCKcre^{-/-} MSTN^{+/+}, MCKcre^{-/-} MSTN^{+/flox}, and MCKcre^{-/-} MSTN^{flox/flox} F2 mice. There was no evidence in any of the analysed traits for an effect of MSTN genotype within the MCKcre^{-/-} genotype (FIG. 15 and FIG. 20). In FIG. 15, Animals are sorted by sex, MCKcre genotype (circles: MCKcre^{-/-}, diamonds: MCKcre^{+/?}), and MSTN genotype. The shaded symbols correspond to individual measurements, the white symbols to the average of the corresponding genotype class. Altogether these results indicate that the functionality of the MSTN^{flox} allele is essentially equivalent to that of its wild type counterpart.

Next it was examined whether the MCKcre transgene might on its own have an effect on the examined phenotypes. This was achieved by comparing the phenotypes of MCKcre^{+/?} MSTN^{+/+} versus MCKcre^{-/-} MSTN^{-/-} mice. Although not statistically significant sensu stricto (p≧0.07), it is noteworthy that was some evidence for a slightly deleterious effect of the MCKcre^{+/?} genotype on live weight, carcass weight, as well as on the weight of several individual organs (FIG. 15 and FIG. 20). If not an artifact, this can be either due to the expression of cre in skeletal muscle, or be an effect of the transgene insertion, or of a QTL allele linked to the transgene insertion site.

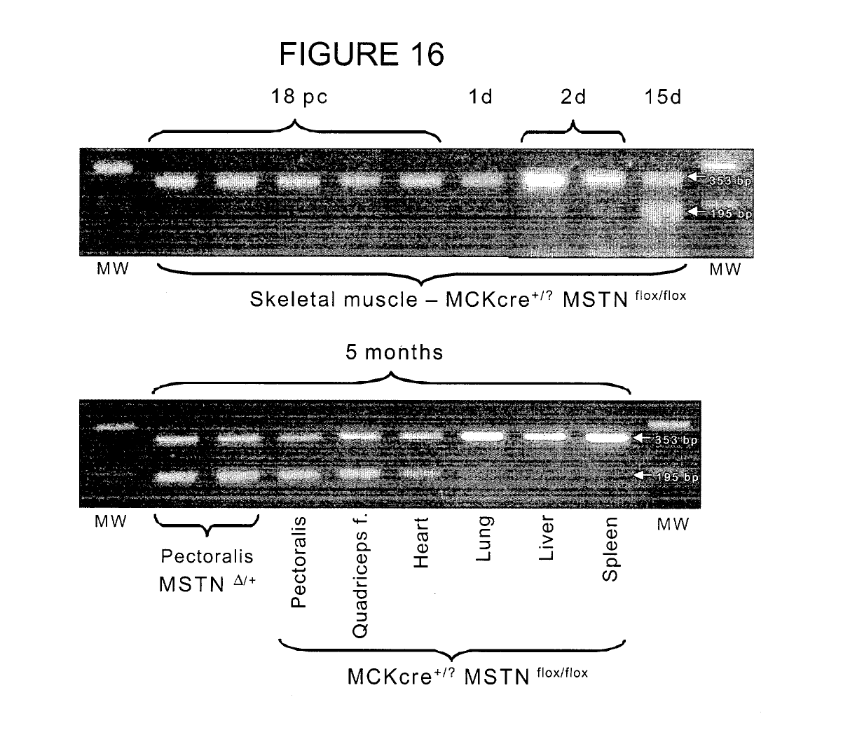

The MCKcre transgene induces a post-natal, muscle-specific inactivation of the MSTN gene. To study the spatio-temporal pattern of cre mediated excision of the third exon of the MSTN^{flox} allele a PCR assay was developed that allowed for the co-amplification of (i) a “control” 335 base pair fragment located approximately 3 Kb upstream of exon I, and (ii) a 195 base pair fragment from a MSTN^{flox} allele having undergone a cre-mediated deletion of exon III. The distance separating the latter primer pair is too large (>4 Kb) to allow amplification of the corresponding PCR products from the intact MSTN^{flox} allele or the wild type MSTN⁺ allele (FIG. 12). The assay was calibrated on genomic DNA extracted from MSTN^{+/+}, MSTN^{Δ/+}, MSTN^{Δ/Δ} mice, as well as on samples comprising varying proportions of MSTN^{Δ/Δ} and MSTN^{+/+} DNA (1/1 to 1/63). This indicated that an excision rate ≦3% could reliably be detected. This assay was applied to genomic DNA extracted from a range of tissues of five month old MCKcre^{+/?} MSTN^{flox/flox} animals. As expected, the Δ-specific 195 base pair fragment was detected in skeletal muscle and cardiac muscle, but not in any of the other tissues that were analysed (kidney, lung, liver, spleen and testis) (FIG. 16). The intensity ratios of the 353 base pair and 195 base pair fragments indicated that at least 50% of the MSTN^{flox} molecules have undergone cre-mediated deletion in skeletal muscle, and slightly less in the heart. As myotubes only contribute approximately half of the nuclei from skeletal muscle (Schmalbruch et al. Anat. Rec. 189:169-175 1977) it was speculated that the excision rate in myotubes may be essentially complete.

Next the test was applied to genomic DNA extracted from skeletal muscle of MCkcre^{+/?} MSTN^{flox/flox} 18 days post coitum foetuses, as well as one, two and fifteen day old young. The Δ-specific 195 base pair fragment was absent in the 18 day post coitum foetuses as well as in the one and two day old newborns. This indicates that the vast majority of the MSTN^{flox} alleles would still be intact and hence functional at these developmental stages. The 195 base pair fragment was, however, present in the 15 day old young, at levels similar to those found in five-month old animals (FIG. 16). In FIG. 16, the developmental stage/age of the analyzed animals is indicated above the gel images, while the analyzed tissue and genotype of the individuals are indicated below. The lanes marked “MW” correspond to the molecular weight marker. The 353 base pair amplification product serves as an internal control and corresponds to a segment of the MSTN promoter. The 195 base pair amplicon is specific for the MSTN^{flox} allele having undergone cre-mediated deletion of exon III (FIG. 12).

Next the effect of the cre-mediated excision of MSTN expression was examined using PCR. No MSTN specific RT-PCR products could be amplified from skeletal muscle of two month old MCKcre^{+/?} MSTN^{flox/flox} mice, contrary to what was observed in MCKcre^{./.} MSTN^{flox/flox} and in MCKcre^{-/-} MSTN^{+/+}, but identical to the situation in constitutive MSTN knock outs (MSTN^{Δ/Δ} FIG. 14). This strongly suggests that the excision rate is indeed essentially complete in myotube nuclei.

These experiments have shown that post-natal, muscle-specific inactivation of the MSTN gene causes a “doublemuscling” phenotype. Monitoring growth in the intercross population clearly revealed a major effect of the MSTN^{./.} genotype within the MCKcre^{+/?} sub-population (FIG. 20 and FIG. 15). At two months of age, MCKcre^{+/?} MSTN^{flox/flox} animals of both sexes weighted on average 5.6 grams more (+23%; p<0.0001) than their MCKcre^{+/?} MSTN^{+/+}counterparts. At five months of age, this difference had increased to 7.9 grams (+26%; p<0.0001). A modest effect was detectable in MCKcre^{+/?} MSTN^{flox/+} individuals as well, weighing 2.2 grams more (+9%; p<0.05) at two months and 3 grams more (+10%; p<0.001) at five 5 months.

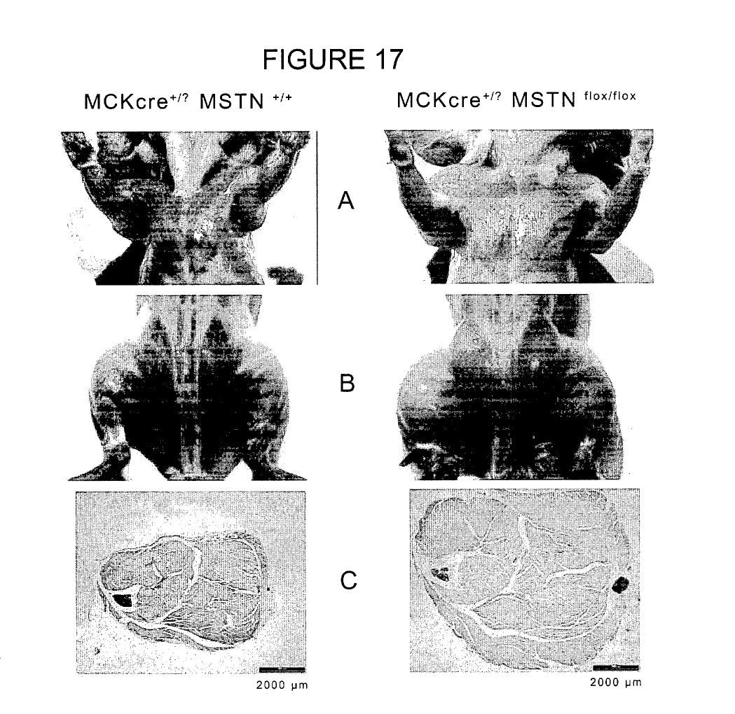

Visual examination of the carcasses of MCKcre^{+/?} MSTN^{flox/flox} animals revealed a marked, generalized muscular hypertrophy. (FIGS. 17A-C). This impression was quantified by showing that the carcasses of MCKcre^{+/?} MSTN^{flox/flox} and MCKcre^{+/?} MSTN^{flox/+} animals weighted on average 6.3 grams (+37%; p<0.0001) and 1.9 grams (+11%; p<0.001) more than that of their MCKcre^{+/?} MSTN^{+/+}counterparts (FIG. 20 and FIG. 15). The increase in carcass weight therefore accounted for 80% and 64% of the increase in weight of MCKcre^{+/?} MSTN^{flox/flox} and MCKcre^{+/?} MSTN^{flox/+} animals respectively. This increase in carcass weight was shown to be primarily due to an increase in muscle mass by (i) comparing the weight of the pectoralis muscle, and (ii) comparing the cross-sectional area of lower leg muscles. When compared to MCKcre^{+/?} MSTN^{+/+}animals, the weight of the pectoralis was shown to be increased by 77% (p<0.0001) in MCKcre^{+/?} MSTN^{flox/flox} and 14% (p<0.05) in MCKcre^{+/?} MSTN^{flox/+} animals (FIG. 20). The total cross-sectional area of the lower leg was shown to be increased by 74%. (p<0.05) in MCKcre^{+/?} MSTN^{flox/flox} animals (FIGS. 17A-C and FIG. 20). Superficial muscle layers seemed to be more profoundly affected than the deeper muscle layers. Indeed the cross-sectional area of the external biceps femoris group and internal adductor group of muscles (FIG. 13) were respectively increased two (+209%) and five-fold (+566%) in MCKcre^{+/?} MSTN^{flox/flox} animals, while the areas occupied by the deeper laying anterior tibialis cranialis group and posterior gastrocnemius plantaris group were increased by only 33% and 65% respectively (FIG. 20). It is noteworthy that the weight of several internal organs (particularly liver, lung and to a lesser extent heart) was also increased in MCKcre+/? MSTN^{flox/flox} when compared to MCKcre^{+/?} MSTN^{+/+} animals (FIG. 20). Taken together, these results clearly demonstrate that post-natal inactivation of MSTN in skeletal muscle still has a major effect on muscle growth in the mouse.

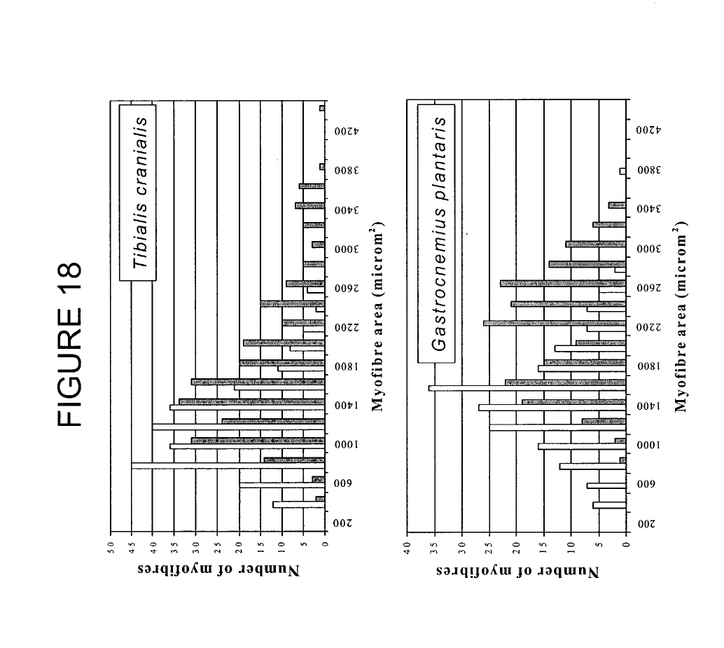

Next in order to determine whether the observed muscular hypertrophy resulted from an increase in the number (hyperplasia) and/or an increase in the size of individual myotubes, the cross sectional area of individual muscle fibers was measured in the tibialis cranialis and gastrocnemius plantaris group of muscles in MCKcre^{+/?} MSTN^{+/+}, MCKcre^{+/?} MSTN^{flox/+} and MCKcre^{+/?} MSTN^{flox/flox} individuals. FIG. 18 shows the frequency distribution of individual fibre area in the MCKcre^{+/?} MSTN^{+/+}and MCKcre^{+/?} MSTN^{flox/flox} genotypes. In MCKcre^{+/?} MSTN^{flox/flox} individuals, average myofibre area is increased by 52% (p<0.0001) in the tibialis cranialis muscle group and by 49% (p<0.0001) in the gastrocnemius plantaris muscle group, when compared to the MCKcre^{+/?} MSTN^{+/+}genotype (FIG. 20). It is noteworthy that in MCKcre^{+/?} MSTN^{flox/flox} individuals, individual fibre area seems to be distributed bimodally (FIG. 18). Comparing the effect on the area of the muscle group with that on the area of individual muscle fibres within the corresponding muscle group indicates that the hypertrophy of individual myotubes accounts entirely for the increase in muscle mass of the tibialis cranialis group, while accounting for 71% of the increase in muscle mass of the gastrocnemius plantaris group, the remainder reflecting myofibre hyperplasia.

All patents and publications mentioned in this specification are indicative of the levels of those skilled in the art to which the invention pertains. All patents and publications are herein incorporated by reference to the same extent as if each individual publication was specifically and individually indicated to be incorporated by reference.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and drawings/figures.

One skilled in the art will readily appreciate that the instant invention is well adapted to carry out the objectives and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which is encompassed within the spirit of the invention and are defined by the scope of the appended claims. Although the instant invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

What is claimed is:

1. A mouse embryonic stem cell comprising a floxed myostatin allele, wherein said floxed myostatin allele comprises, in order from 5′ to 3′:
a) exon 1 of the myostatin gene;
b) exon 2 of the myostatin gene;
c) a first loxP site;
d) exon 3 of the myostatin gene; and
e) a second loxP site, wherein the floxed myostatin allele is integrated in the genome of a mouse produced using the embryonic stem cell.

2. A transgenic mouse comprising a myostatin gene wherein exon 3 of said myostatin gene is floxed, and wherein each allele of said myostatin gene comprises, in order from 5′ to 3′:
a) exon 1 of the myostatin gene;
b) exon 2 of the myostatin gene;
c) a first loxP site;
d) exon 3 of the myostatin gene; and
e) a second loxP site, wherein each allele is integrated into the genome of the transgenic mouse.

3. The transgenic mouse of claim 2 wherein the myostatin gene of said transgenic mouse can be conditionally inactivated with excision of exon 3 by cre recombinase.

4. A method to produce a transgenic mouse having a myostatin gene conditionally inactivated by excision of exon 3, the method comprising the steps of:
a) intercrossing a male transgenic mouse of claim 2 with a female transgenic mouse of claim 2;
b) expressing a nucleotide sequence encoding the cre recombinase in the zygotes resulting from the intercross of step (a);
c) selecting the offspring resulting from step (b) for the cre-mediated excision of exon 3; and
(d) intercrossing the offspring selected in step (c) to produce a transgenic mouse having a having a myostatin gene conditionally inactivated by excision of exon 3.

5. A transgenic mouse produced by the method of claim 4.

6. The transgenic mouse of claim 5 having a phenotype characterized by skeletal muscle hypertrophy.


SEQUENCE LISTING (123 sequences)

SEQ ID NO: 1 - DNA, 38 bp, Artificial
  primer sequence
gggcgcgccggatcctctcgtgagtctagagtcgactg

SEQ ID NO: 2 - DNA, 39 bp, Artificial
  primer sequence
ccttttaaattaaattaattaatttaaatcaatctaaag

SEQ ID NO: 3 - DNA, 27 bp, Artificial
  primer sequence
catcattggaaaacgctcttcggggcg

SEQ ID NO: 4 - DNA, 36 bp, Artificial
  primer sequence
cctataaaaatagcggtatcaggaggccctttcgtc

SEQ ID NO: 5 - DNA, 34 bp, Artificial
  primer sequence
gttcgatgtaacccacgcgtgcacccaactgatc

SEQ ID NO: 6 - DNA, 35 bp, Artificial
  primer sequence
cccctggaagctccctcctgcgctctcctgttccg

SEQ ID NO: 7 - DNA, 26 bp, Artificial
  adaptor sequence
ctctgtcgactgtcttctattcctca

SEQ ID NO: 8 - DNA, 34 bp, Artificial
  adaptor sequence
gtacgagacagctgacagaagataaggagtttaa

SEQ ID NO: 9 - DNA, 30 bp, Artificial
  adaptor sequence
tcgagagtcttctattccgtcttctcctca

SEQ ID NO: 10 - DNA, 30 bp, Artificial
  adaptor sequence
ctcagaagattaggcagaagaggagtttaa

SEQ ID NO: 11 - DNA, 20 bp, Artificial
  adaptor sequence
taggggttgcggccgcttgg

SEQ ID NO: 12 - DNA, 20 bp, Artificial
  adaptor sequence
ccaacgccggcgaaccgcgc

SEQ ID NO: 13 - DNA, 15 bp, Artificial
  adaptor sequence
ccaagcggccgcaac

SEQ ID NO: 14 - DNA, 23 bp, Artificial
  adaptor sequence
tcgaggttcgccggcgttgatcc

SEQ ID NO: 15 - DNA, 21 bp, Artificial
  adaptor sequence
ctgacgagaccctattcctgg

SEQ ID NO: 16 - DNA, 21 bp, Artificial
  adaptor sequence
gctctgggataaggaccgcgc

SEQ ID NO: 17 - DNA, 32 bp, Artificial
  adaptor sequence
tcgacgtcctcgtgcttggcgcgccctgtctc

SEQ ID NO: 18 - DNA, 32 bp, Artificial
  adaptor sequence
gcaggagcacgaaccgcgcgggacagagttaa

SEQ ID NO: 19 - DNA, 30 bp, Artificial
  adaptor sequence
cgcggactgtctctgctgtctattcctcac

SEQ ID NO: 20 - DNA, 30 bp, Artificial
  adaptor sequence
ctgacagagacgacagataaggagtgttaa

SEQ ID NO: 21 - DNA, 42 bp, Artificial
  adaptor sequence
ggccctataacttcgtatagcatacattatacgaagttatgc

SEQ ID NO: 22 - DNA, 42 bp, Artificial
  adaptor sequence
gatattgaagcatatcgtatctaatatgcttcaatacgccgg

SEQ ID NO: 23 - DNA, 46 bp, Artificial
  adaptor sequence
tcgagctataacttcgtatagcatacattatacgaagttatggaca

SEQ ID NO: 24 - DNA, 46 bp, Artificial
  adaptor sequence
cgatattgaagcatatcgtatgtaatatgcttcaatacctgtaatt

SEQ ID NO: 25 - DNA, 43 bp, Artificial
  adaptor sequence
ttaacttataacttcgtatagcatacattatacgaagttatgc

SEQ ID NO: 26 - DNA, 43 bp, Artificial
  adaptor sequence
gaatattgaagcatatcgtatgtaatatgcttcaatacgagct

SEQ ID NO: 27 - DNA, 26 bp, Artificial
  primer sequence
cacacaggaaacagctatgaccatga

SEQ ID NO: 28 - DNA, 25 bp, Artificial
  primer sequence
actgcccacttggcagtacatcaag

SEQ ID NO: 29 - DNA, 25 bp, Artificial
  primer sequence
taactagagaacccactgcttactg

SEQ ID NO: 30 - DNA, 25 bp, Artificial
  primer sequence
ttccgagacaatcgcgaacatctac

SEQ ID NO: 31 - DNA, 25 bp, Artificial
  primer sequence
cgagcggcttgacctggctatgctg

SEQ ID NO: 32 - DNA, 25 bp, Artificial
  primer sequence
caccccaggctccataccgacgatc

SEQ ID NO: 33 - DNA, 25 bp, Artificial
  primer sequence
tgcggtgggctctatggcttctgag

SEQ ID NO: 34 - DNA, 25 bp, Artificial
  primer sequence
cttgttccaaactggaacaacactc

SEQ ID NO: 35 - DNA, 30 bp, Artificial; identical to SEQ ID NO: 9
  primer sequence

SEQ ID NO: 36 - DNA, 25 bp, Artificial
  primer sequence
gaggaggtatgaatgtcatttcaac

SEQ ID NO: 37 - DNA, 25 bp, Artificial
  primer sequence
tattcctttcataccctaactcaac

SEQ ID NO: 38 - DNA, 25 bp, Artificial
  primer sequence
agctgattatccatgcttttcatag

SEQ ID NO: 39 - DNA, 25 bp, Artificial
  primer sequence
cattaaagttcttgcagtgtagtag

SEQ ID NO: 40 - DNA, 25 bp, Artificial
  primer sequence
agtggaagaaattctctcttcactc

SEQ ID NO: 41 - DNA, 25 bp, Artificial
  primer sequence
ctcgacagcacagaattcatgaatg

SEQ ID NO: 42 - DNA, 25 bp, Artificial
  primer sequence
gtagctcacctcaccctgcatgttc

SEQ ID NO: 43 - DNA, 25 bp, Artificial
  primer sequence
acaaccatatttttagaatgctgtg

SEQ ID NO: 44 - DNA, 25 bp, Artificial
  primer sequence
tcagctctgactttatgaacaaatg

SEQ ID NO: 45 - DNA, 25 bp, Artificial
  primer sequence
ccagctacccagattccccactgag

SEQ ID NO: 46 - DNA, 25 bp, Artificial
  primer sequence
aaagagcaagcccttctgcttcaag

SEQ ID NO: 47 - DNA, 25 bp, Artificial
  primer sequence
gcaatataagtagctaaatgtagtc

SEQ ID NO: 48 - DNA, 25 bp, Artificial
  primer sequence
aagagggccagatcacctcagggtg

SEQ ID NO: 49 - DNA, 25 bp, Artificial
  primer sequence
tattagagcaggcctataaagtcag

SEQ ID NO: 50 - DNA, 25 bp, Artificial
  primer sequence
tttgttcagctctttaagagttcac

SEQ ID NO: 51 - DNA, 25 bp, Artificial
  primer sequence
ctcctgtttgggaagctgaggagtc

SEQ ID NO: 52 - DNA, 25 bp, Artificial
  primer sequence
tgacagtaaagtgcaatctgtgttc

SEQ ID NO: 53 - DNA, 25 bp, Artificial
  primer sequence
ttatctactcggcctaagtacagag

SEQ ID NO: 54 - DNA, 25 bp, Artificial
  primer sequence
tgcgttaagtgctgggtaattagag

SEQ ID NO: 55 - DNA, 25 bp, Artificial
  primer sequence
caagagttttacagagattaataag

SEQ ID NO: 56 - DNA, 25 bp, Artificial
  primer sequence
taaaaccctgtctgtcacaagtcac

SEQ ID NO: 57 - DNA, 27 bp, Artificial
  primer sequence
cggacggtacatgcactaatatttcac

SEQ ID NO: 58 - DNA, 24 bp, Artificial
  primer sequence
atcattttaaaaatcagcacaatc

SEQ ID NO: 59 - DNA, 25 bp, Artificial
  primer sequence
gctgcgcctggaaacagctcctaac

SEQ ID NO: 60 - DNA, 27 bp, Artificial
  primer sequence
tcactgctgtcatccctctggacgtcg

SEQ ID NO: 61 - DNA, 25 bp, Artificial
  primer sequence
tatatctgttaaagtatatcaacag

SEQ ID NO: 62 - DNA, 25 bp, Artificial
  primer sequence
atttcattgtcggtatgtttctcag

SEQ ID NO: 63 - DNA, 25 bp, Artificial
  primer sequence
ctataatgtaaggactgtgagattc

SEQ ID NO: 64 - DNA, 25 bp, Artificial
  primer sequence
tattaaatgcattatcatgagccac

SEQ ID NO: 65 - DNA, 25 bp, Artificial
  primer sequence
acagaaatctttcgtgttctgcctg

SEQ ID NO: 66 - DNA, 25 bp, Artificial
  primer sequence
acatttcaggcagttcctgtttgag

SEQ ID NO: 67 - DNA, 25 bp, Artificial
  primer sequence
ggaaaagcaattgttagtgctgaac

SEQ ID NO: 68 - DNA, 26 bp, Artificial
  primer sequence
ctccagactgactggtacagctgctc

SEQ ID NO: 69 - DNA, 25 bp, Artificial
  primer sequence
ttctgaactatgaatgaagttccac

SEQ ID NO: 70 - DNA, 25 bp, Artificial
  primer sequence
acagtgtttgtgcaaatcctgagac

SEQ ID NO: 71 - DNA, 25 bp, Artificial
  primer sequence
caatgcctaagttggattcaggctg

SEQ ID NO: 72 - DNA, 25 bp, Artificial
  primer sequence
ataagccagacaaagtatcttactc

SEQ ID NO: 73 - DNA, 24 bp, Artificial
  primer sequence
tgaaaaatgttggttcacataaag

SEQ ID NO: 74 - DNA, 24 bp, Artificial
  primer sequence
ctatatacatatcatggcttcaac

SEQ ID NO: 75 - DNA, 25 bp, Artificial
  primer sequence
tagtgagtcagtgataggacaagac

SEQ ID NO: 76 - DNA, 25 bp, Artificial
  primer sequence
attgaacttgggaatatacagtctg

SEQ ID NO: 77 - DNA, 25 bp, Artificial
  primer sequence
aaggaatatcacactaaccaccttg

SEQ ID NO: 78 - DNA, 25 bp, Artificial
  primer sequence
gtggttagtgtgatattccttagag

SEQ ID NO: 79 - DNA, 25 bp, Artificial
  primer sequence
tatacatacagccactgtcatcatg

SEQ ID NO: 80 - DNA, 25 bp, Artificial
  primer sequence
tgctattatgtctgataatagtatg

SEQ ID NO: 81 - DNA, 21 bp, Artificial
  primer sequence
tcccggagagactttgggctt

SEQ ID NO: 82 - DNA, 25 bp, Artificial
  primer sequence
tgggtgtgtctgtcaccttgacttc

SEQ ID NO: 83 - DNA, 25 bp, Artificial
  primer sequence
ccccctcacggtcgattttgaagcc

SEQ ID NO: 84 - DNA, 23 bp, Artificial
  primer sequence
tcccatccaaaggcttcaaaatc

SEQ ID NO: 85 - DNA, 25 bp, Artificial
  primer sequence
cccattaatatgctatattttaatg

SEQ ID NO: 86 - DNA, 26 bp, Artificial
  primer sequence
gagcacccacagcggtctactaccat

SEQ ID NO: 87 - DNA, 25 bp, Artificial
  primer sequence
gtagaccgctgtgggtgctcatgag

SEQ ID NO: 88 - DNA, 25 bp, Artificial
  primer sequence
tggtctgctgagttaggagggtatg

SEQ ID NO: 89 - DNA, 25 bp, Artificial
  primer sequence
tacaaaggctacatatagattcttc

SEQ ID NO: 90 - DNA, 25 bp, Artificial
  primer sequence
cggaagaatctatatgtagcctttg

SEQ ID NO: 91 - DNA, 25 bp, Artificial
  primer sequence
gcacagcgggagtgactgctgcatc

SEQ ID NO: 92 - DNA, 25 bp, Artificial
  primer sequence
aatgtattgtactcatagctaaatg

SEQ ID NO: 93 - DNA, 25 bp, Artificial
  primer sequence
aataatttcatttagctatgagtac

SEQ ID NO: 94 - DNA, 25 bp, Artificial
  primer sequence
catggtggctgtatctatgaatgtg

SEQ ID NO: 95 - DNA, 25 bp, Artificial
  primer sequence
aattggcagtggtatatactcctag

SEQ ID NO: 96 - DNA, 25 bp, Artificial
  primer sequence
ctaccttcatcaggtcagggatgtg

SEQ ID NO: 97 - DNA, 26 bp, Artificial
  primer sequence
gggctgaccgcttcctcgtgctttac

SEQ ID NO: 98 - DNA, 25 bp, Artificial
  primer sequence
catcgccatgggtcacgacgagatc

SEQ ID NO: 99 - DNA, 25 bp, Artificial
  primer sequence
gggcaccggacaggtcggtcttgac

SEQ ID NO: 100 - DNA, 25 bp, Artificial
  primer sequence
cattccaggcctgggtggagaggct

SEQ ID NO: 101 - DNA, 25 bp, Artificial
  primer sequence
aattgtgacatgataaaaatccatc

SEQ ID NO: 102 - DNA, 25 bp, Artificial
  primer sequence
ttttgatggatttttatcatgtcac

SEQ ID NO: 103 - DNA, 25 bp, Artificial
  primer sequence
tgtgtcttagacctcaatggccatg

SEQ ID NO: 104 - DNA, 25 bp, Artificial
  primer sequence
gtacattagaatggatggtttgcag

SEQ ID NO: 105 - DNA, 25 bp, Artificial
  primer sequence
tttgttgttctcagatttctgtggc

SEQ ID NO: 106 - DNA, 25 bp, Artificial
  primer sequence
ctaaccactccaaatcactctgttc

SEQ ID NO: 107 - DNA, 25 bp, Artificial
  primer sequence
cttaatgtccctgggagcagatctg

SEQ ID NO: 108 - DNA, 25 bp, Artificial
  primer sequence
tcagtccctgacaatacagtcactg

SEQ ID NO: 109 - DNA, 25 bp, Artificial
  primer sequence
gtcaggtgtggtagcctagaaatgc

SEQ ID NO: 110 - DNA, 25 bp, Artificial
  primer sequence
tttgctttgatgatagtgaagcgtc

SEQ ID NO: 111 - DNA, 25 bp, Artificial
  primer sequence
ggagtgaacaaacactgagttccag

SEQ ID NO: 112 - DNA, 25 bp, Artificial
  primer sequence
taaactgcccatagacagtgtattg

SEQ ID NO: 113 - DNA, 25 bp, Artificial
  primer sequence
catccagctcagcctatgtgttgag

SEQ ID NO: 114 - DNA, 25 bp, Artificial
  primer sequence
tgtaaggatgattagaaatgacaac

SEQ ID NO: 115 - DNA, 30 bp, Artificial; identical to SEQ ID NO: 19
  primer sequence

SEQ ID NO: 116 - DNA, 30 bp, Artificial
  primer sequence
aattgtgaggaatagacagcagagacagtc

SEQ ID NO: 117 - DNA, 32 bp, Artificial; identical to SEQ ID NO: 17
  primer sequence

SEQ ID NO: 118 - DNA, 24 bp, Artificial
  primer sequence
cgacgttgtaaaacgacggccagt

SEQ ID NO: 119 - DNA, 25 bp, Artificial
  primer sequence
ccatatagtgctcagaaagagctac

SEQ ID NO: 120 - DNA, 25 bp, Artificial
  primer sequence
tgggctaattatgaattattcactc

SEQ ID NO: 121 - DNA, 22 bp, Artificial
  primer sequence
agactcctacaacagtgtttgt

SEQ ID NO: 122 - DNA, 25 bp, Artificial
  primer sequence
accttcaacaccccagccatgtacg

SEQ ID NO: 123 - DNA, 25 bp, Artificial
  primer sequence
ctgatccacatctgctggaaggtgg